(19) United States
(12) Patent Application Publication
(10) Pub. No.: US 2026/0297181 A1
(43) Pub. Date: Oct. 1, 2026

(54) ENGINEERED ANTIBODY FUSION PROTEINS WITH MODULABLE BINDING CONSTANTS AND THEIR APPLICATIONS

(71) Applicant: THE UNIVERSITY OF NORTH CAROLINA AT CHAPEL HILL, Chapel Hill, NC (US)

(72) Inventors: Koji SODE, Chapel Hill, NC (US); Ellie WILSON, Chapel Hill, NC (US)

(21) Appl. No.: 19/490,841

(22) PCT Filed: Jun. 7, 2024
(86) PCT No.: PCT/US2024/033094

§ 371 Date: Dec. 5, 2025

Related U.S. Application Data
(60) Provisional application No. 63/506,971, filed on Jun. 8, 2023.

(51) Int. Cl. C07K 16/26; C07K 14/415; G01N 33/543; G01N 33/74

(52) U.S. Cl. CPC C07K 16/26; C07K 14/415; G01N 33/5438; G01N 33/74; C07K 2317/40; C07K 2317/622; C07K 2317/92; C07K 2319/00; G01N 2333/62

(57) ABSTRACT

Compositions and methods of making engineered antibody fusion proteins with redox-sensitive domains with modulable binding affinity and devices for the continuous in vivo detection of target molecules are described.

### CROSS REFERENCE TO RELATED APPLICATIONS

This application claims the benefit of priority from U.S. Provisional Application No. 63/506,971, filed Jun. 8, 2023, which is hereby incorporated by reference in its entirety.

### REFERENCE TO A SEQUENCE LISTING

The Sequence Listing written in file name 614159SEQLIST.xml is 55.8 kilobytes, was created on Jun. 7, 2024, and is hereby incorporated by reference.

### BACKGROUND

Sensors capable of continuously monitoring biologically relevant molecules such as metabolites, hormones, and therapeutic compounds in vivo would provide highly useful medical information which could improve treatments and outcomes for many patients. However, clinically useful continuous sensors are only available for glucose monitoring, employing either an enzyme, such as glucose oxidase, or an abiotic synthetic receptor harboring a fluorescent probe. For continuous monitoring, the sensing molecule must be regenerated after the recognition of its target, which has limited the development of continuous sensors, particularly for hormones, such as insulin.

Antibodies can be generated to selectively bind to a wide range of biologically relevant molecules and can be used for detection and quantification of their antigens through a variety of immunoassays. The high binding affinity that antibodies typically have for their antigens (e.g., K_{d} ranges 10⁻⁸ to 10⁻¹²) enables high selectivity and sensitivity, but also keeps antibodies tightly and irreversibly bound to their antigen under physiological conditions. Thus, the ligand binding site of an antibody can only be regenerated under harsh physical or chemical conditions (e.g., extreme changes in pH, temperature, or denaturing conditions). Due to these characteristics, the use of antibodies for the detection of targets such as insulin has only been performed in in vitro immunoassays, and antibodies have been used for single use monitoring, not for continuous biosensing applications in vivo. The antibody fusion proteins disclosed herein address the need for antibody-based biosensors capable of continuously monitoring targets in vivo and in situ.

### BRIEF SUMMARY

Compositions and methods of making engineered antibody fusion proteins with redox-sensitive domains with modulable binding affinity are provided and their application in devices for the continuous in vivo detection of target molecules.

One embodiment is an engineered antibody fusion protein, the protein comprising a variable region of an immunoglobulin heavy chain (V_{H}), a variable region of an immunoglobulin light chain (V_{L}), and a redox-sensitive domain, wherein the redox-sensitive domain is capable of undergoing a conformational change upon exposure to an external signal, and wherein the conformational change modulates an affinity of the antibody fusion protein for a target molecule upon exposure to the external signal.

In some embodiments, the amino acid sequence of the redox-sensitive domain is located at the C-terminal end of the antibody fusion protein.

In other embodiments, the amino acid sequence of the redox-sensitive domain is located at the N-terminal end of the antibody fusion protein.

In some embodiments, the V_{H} region and the V_{L} region are adjacent and comprise a single chain variable region (scFv), optionally wherein a linker sequence is between the amino acid sequence of the V_{H} region and the amino acid sequence of the V_{L} region, wherein the linker sequence comprises one or more glycine residues.

In other embodiments, the amino acid sequence of the redox-sensitive domain is located between the amino acid sequence of the V_{H} region and the amino acid sequence of the V_{L} region. In some embodiments, the amino acid sequence of the V_{H} region is located at the C-terminal end of the antibody fusion protein and the amino acid sequence of the V_{L} region is located at the N-terminal end of the antibody fusion protein. In some embodiments, the amino acid sequence of the V_{H} region is located at the N-terminal end of the antibody fusion protein and the amino acid sequence of the V_{L} region is located at the C-terminal end of the antibody fusion protein.

In embodiments, the target molecule is a metabolite, a hormone, and/or a therapeutic compound. In some embodiments, the target molecule is selected from the group consisting of insulin, glucagon, and glucagon-like peptide 1. In one embodiment, the target molecule is insulin and/or insulin analogues.

In some embodiments, the V_{H} region and the V_{L} region are anti-insulin.

In some embodiments, the scFv comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 1. In one embodiment, the scFv comprises an amino acid sequence set forth in SEQ ID NO: 1.

In embodiments, the redox-sensitive domain comprises a heme cofactor or a flavin cofactor. In some embodiments, the redox-sensitive domain is selected from the group consisting of a bacterium-derived cytochrome c-like molecule (CYTc) domain, a light-oxygen-voltage-sensing (LOV) domain, and a cGMP-specific phosphodiesterases, adenylyl cyclases, FhlA (GAF) domain. In some embodiments, wherein the LOV domain is a second LOV domain of a phototropin (LOV2). In one embodiment, the LOV2 domain is derived from Avena sativa (asLOV2).

In some embodiments, the redox-sensitive domain comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 2 or SEQ ID NO: 36. In one embodiment, the redox-sensitive domain comprises an amino acid sequence set forth in SEQ ID NO: 2 or SEQ ID NO: 36.

In some embodiments, the amino acid sequence of the V_{H} region comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 6. In one embodiment, the amino acid sequence of the V_{H} region comprises an amino acid sequence set forth in SEQ ID NO: 6.

In some embodiments, the amino acid sequence of the V_{L} region comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 7. In one embodiment, the amino acid sequence of the V_{L} region comprises an amino acid sequence set forth in SEQ ID NO: 7.

In some embodiments, the antibody fusion protein comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to any one of SEQ ID NOs: 3-5.

In some embodiments, the antibody fusion protein comprises an amino acid sequence set forth in any one of SEQ ID NO: 3-5 and 16-20.

In embodiments, a sensitivity towards the target molecule is modulated by exposure to the external signal. In some embodiments, the sensitivity of the antibody fusion protein towards the target molecule is at least 2-fold, at least 5-fold, at least 10-fold, at least 100-fold, or at least 1000-fold different upon exposure to the external signal. In embodiments, a limit of detection (LOD) of the target molecule is modulated by exposure to the external signal. In embodiments, a specific binding ability of the antibody fusion protein towards the target molecule is modulated by exposure to the external signal. In some embodiments, the specific binding ability of the antibody fusion protein towards the target molecule is at least 2-fold, at least 5-fold, at least 10-fold, at least 100-fold, or at least 1000-fold different upon exposure to the external signal.

In embodiments, the dissociation constant (K_{d}) of the antibody fusion protein towards the target molecule is modulated by exposure to the external signal. In some embodiments, the K_{d} a of the antibody fusion protein is at least 10, at least 10², at least 10³, at least 10⁴, at least 10⁵, at least 10⁶, at least 10⁷, at least 10⁸, at least 10⁹, or at least 10¹⁰ times different upon exposure to the external signal.

In embodiments, the external signal is redox potential or light. In some embodiments, the external signal is blue light. In some embodiments, the light has a wavelength of 400-700 nm.

Another embodiment is an electrode comprising said antibody fusion protein immobilized on an electroconductive material.

A further embodiment is a device for detection of the target molecule, comprising said engineered antibody fusion protein. In some embodiments, the detection is continuous. In some embodiments, the detection is performed in vivo. In some embodiments, the target molecule detected by said device is insulin.

An embodiment is a polynucleotide encoding said antibody fusion protein. In some embodiments, said polynucleotide comprises a nucleotide sequence having at least 90%, at least 91%, at least 92%, at least 93%, at least 94%, at least 95%, at least 96%, at least 97%, at least 98%, at least 99%, at least 99.5%, or at least 99.9% sequence identity to any one of SEQ ID NOs: 8-10 and 31-35. In some embodiments, said polynucleotide comprises a nucleotide sequence set forth in any one of SEQ ID NOs: 8-10 and 31-35.

Another embodiment is a recombinant plasmid comprising a promoter operably linked to said polynucleotide sequence and prepared by inserting the nucleotide sequence into an expression vector. In some embodiments, the expression vector is a pET30c vector.

A further embodiment is a method of producing a recombinant antibody fusion protein, the method comprising: i) introducing said recombinant plasmid into a host cell and ii) expressing the antibody fusion protein. In some embodiments, the host cell is E. coli.

### BRIEF DESCRIPTION OF FIGURES

FIG. 1A shows the structure of a redox-sensitive light-oxygen-voltage-sensing domain derived from Avena sativa (asLOV2) and FIG. 1B shows its flavin mononucleotide (FMN) cofactor, which binds to the Cys966 upon excitation with blue light, which leads to the undocking of the Jα-helix.¹

FIG. 2 shows the structural model of an engineered antibody fusion protein with an anti-insulin single-chain variable fragment (scFV) domain and an asLOV2 domain at the C-terminus (cLOV) as predicted by AlphaFold 2.

FIG. 3 shows a recombinant plasmid for the expression of an antibody fusion protein with an anti-insulin scFV domain and an asLOV2 domain at the C-terminus (cLOV) in a pET30c expression vector.

FIG. 4A shows the chromatogram tracking at 280 nm (UV280) and 450 nm (UV450) absorptions, and FIG. 4B shows SDS-PAGE of the eluted soluble antibody fusion protein with an anti-insulin scFV domain and an asLOV2 domain at the C-terminus (cLOV), which was expressed in Escherichia coli Shuffle T7 Express™ cells.

FIG. 5 shows results of an insulin ELISA with the crude soluble extract of the antibody fusion protein with an anti-insulin scFV domain and an asLOV2 domain at the C-terminus (cLOV).

FIGS. 6A-6B show spectroscopic analyses of an asLOV2 protein (FIG. 6A) and the antibody fusion protein (cLOV) with an anti-insulin scFV domain and an asLOV2 domain at the C-terminus (cLOV) (FIG. 6B) in the dark and in the presence of light. The light exposure was performed using an LED array at 470 nm.

FIG. 7A shows a schematic overview of the insulin ELISA used to measure the binding affinity of the antibody fusion protein with an anti-insulin scFV domain and an asLOV2 domain. FIG. 7B shows the results of the ELISA of the antibody fusion protein with an anti-insulin scFV domain and an asLOV2 domain at the C-terminus (cLOV) in the dark and in the presence of light at various insulin concentrations. For the ELISA assays, the entire procedure including all washing and incubation steps were performed either in a dark room, or under an LED array with constant 470 nm light exposure.

FIGS. 8A-8D show further spectroscopic analyses of an asLOV protein (FIGS. 8A-8B) and an antibody fusion protein with an anti-insulin scFV domain and an asLOV2 domain at the C-terminus (cLOV; FIGS. 8C-8D) by measuring the intrinsic fluorescence of the flavin mononucleotide (FMN) cofactor in light and dark conditions. FIGS. 8A and 8C show fluorescence of asLOV2 and the cLOV antibody fusion protein in light and dark conditions. FIGS. 8B and 8D show recovery of the fluorescence signal in the dark at 30 second intervals for two minutes after light exposure.

FIG. 9 shows the structural model of an engineered antibody fusion protein with an anti-insulin single-chain variable fragment (scFV) domain and an asLOV2 domain at the N-terminus (nLOV) as predicted by AlphaFold 2.

FIG. 10 shows a recombinant plasmid for the expression of an antibody fusion protein with an anti-insulin scFV domain and an asLOV2 domain at the N-terminus (nLOV) in a pET30c expression vector.

FIG. 11A shows the chromatogram tracking at 280 nm (UV280) and 450 nm (UV450) absorptions, and FIG. 11B shows SDS-PAGE of the eluted soluble antibody fusion protein with an anti-insulin scFV domain and an asLOV2 domain at the N-terminus (nLOV), which was expressed in E. coli Shuffle T7 Express™ cells.

FIG. 12 shows the intrinsic fluorescence of an antibody fusion protein with an anti-insulin scFV domain and an asLOV2 domain at the N-terminus (nLOV), measured in both dark and light conditions. Recovery is shown by measuring the fluorescence signal in the dark at 30 second intervals for six minutes after light exposure.

FIG. 13 shows the structural model of an antibody fusion protein with an anti-insulin single-chain variable fragment (scFV) domain and a truncated asLOV2 domain at the N-terminus (ntLOV) as predicted by AlphaFold 2.

FIG. 14 shows a recombinant plasmid for the expression of an antibody fusion protein with an anti-insulin scFV domain and a truncated asLOV2 domain at the N-terminus (ntLOV) in a pET30c expression vector.

FIG. 15A shows the chromatogram tracking at 280 nm (UV280) and 450 nm (UV450) absorptions, and FIG. 15B shows SDS-PAGE of the eluted soluble antibody fusion protein with an anti-insulin scFV domain and a truncated asLOV2 domain at the N-terminus (ntLOV), which was expressed in E. coli Shuffle T7 Express™ cells.

FIG. 16 shows the intrinsic fluorescence of an antibody fusion protein with an anti-insulin scFV domain and a truncated asLOV2 domain at the N-terminus (ntLOV), measured in both dark and light conditions. Recovery is shown by measuring the fluorescence signal in the dark at 30 second intervals for six minutes after light exposure.

FIG. 17 shows the structural model of an engineered antibody fusion protein with an asLOV2 domain between an anti-insulin V_{H} region at the N-terminus and an anti-insulin V_{L} region at the C-terminus with glycine-rich linkers (gsLOV) as predicted by AlphaFold 2.

FIG. 18 shows a recombinant plasmid for the expression of an antibody fusion protein with an asLOV2 domain between an anti-insulin V_{H} region at the N-terminus and an anti-insulin V_{L} region at the C-terminus (gsLOV) in a pET30c expression vector.

FIG. 19A shows SDS-PAGE of eluted soluble antibody fusion proteins with an asLOV2 domain between an anti-insulin V_{H} region at the N-terminus and an anti-insulin V_{L} region at the N-terminus (gsLOV) which were expressed in E. coli Shuffle T7 Express™ cells. Variants of the antibody fusion proteins were prepared with mutations to fix the proteins in a “dark state” (C190A) or a “light state” (1272E, A276E). FIGS. 19B-19D show the chromatogram tracking at 280 nm (UV280) and 450 nm (UV450) absorptions for the gsLOV (FIG. 19B), the gsLOV C190A “dark” variant (FIG. 19C), and the gsLOV I272E, A276E “light” variant (FIG. 19D) antibody fusion proteins.

FIG. 20 shows the intrinsic fluorescence of an antibody fusion protein with an asLOV2 domain between an anti-insulin V_{H} region at the N-terminus and an anti-insulin V_{L} region at the N-terminus (gsLOV), measured in both dark and light conditions. Recovery is shown by measuring the fluorescence signal in the dark at 30 second intervals for six minutes after light exposure.

FIG. 21A shows a schematic of an antibody fusion protein binding to insulin as used in a Biolayer Interferometry (BLI) assay. FIGS. 21B-21E show BLI sensor grams for the wild type anti-insulin (FIG. 21B), the gsLOV fusion protein (FIG. 21C), the gsLOV I272E, A276E fusion protein fixed in a “light state” (FIG. 21D), and the gsLOV C190A fusion protein fixed in a “dark state” (FIG. 21E).

FIG. 22 shows the structural model of an engineered antibody fusion protein with an asLOV2 domain between an anti-insulin V_{H} region at the N-terminus and an anti-insulin V_{L} region at the N-terminus without glycine-rich linkers between the domains (nolinkLOV) as predicted by AlphaFold 2.

FIG. 23 shows a recombinant plasmid for the expression of an antibody fusion protein with an asLOV2 domain between an anti-insulin V_{H} region at the N-terminus and an anti-insulin V_{L} region at the N-terminus without glycine-rich linkers between the domains (nolinkLOV) in a pET30c expression vector.

FIG. 24A shows SDS-PAGE of eluted soluble antibody fusion proteins with an asLOV2 domain between an anti-insulin V_{H} region at the N-terminus and an anti-insulin V_{L} region at the N-terminus without glycine-rich linkers between the domains (nolinkLOV) which were expressed in E. coli Shuffle T7 Express™ cells. Variants of the antibody fusion proteins were prepared with mutations to fix the proteins in a “dark state” (C185A) or a “light state” (1267E, A271E). FIGS. 24B-24D show the chromatogram tracking at 280 nm (UV280) and 450 nm (UV450) absorptions for the nolinkLOV (FIG. 24B), the nolinkLOV C185A “dark” variant (FIG. 24C), and the nolinkLOV I267E, A271E “light” variant (FIG. 24D) antibody fusion proteins.

FIGS. 25A-25D show BLI sensor grams for wild type anti-insulin (FIG. 25A), the nolinkLOV fusion protein (FIG. 25B), the nolinkLOV C185A fusion protein fixed in a “dark state” (FIG. 25C), and the nolinkLOV I267E, A271E fusion protein fixed in a “light state” (FIG. 25D).

### DETAILED DESCRIPTION

Antibodies capable of selectively binding their antigens can be easily generated for a vast range of relevant targets and are highly useful in many biomedical applications, including immunoassays. However, the high affinity of an antibody for its antigen (e.g., K_{d} ranges 10⁻⁸ to 10−12) makes the binding essentially irreversible, thus the antibody binding site cannot be regenerated under physiological conditions. These physical properties have made it impossible to use antibodies for continuous in vivo detection of target molecules. The development of an antibody with modulable binding affinity would solve this problem by being able to release the antigen from the antibody upon exposure to an external signal. Disclosed herein are engineered antibody fusion proteins with redox-sensitive domains which undergo a conformational change to modulate binding affinity upon exposure to an external signal.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents and other references mentioned herein are incorporated by reference into their entirety.

### I. Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular aspects only and is not intended to be limiting of the invention. In case of a conflict in terminology, the present specification is controlling.

As used herein, the term “physiological conditions” refers to the range of conditions of temperature, pH, and tonicity (or osmolality) normally encountered within tissues in the body of a living human.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

As used herein, the term “and/or” refers to and encompasses any and all possible combinations of one or more of the associated listed items, as well as the lack of combinations when interpreted in the alternative (“or”).

The term “or” refers to any one member of a particular list.

Unless otherwise apparent from the context, the term “about” encompasses values within a standard margin of error of measurement (e.g., SEM) of a stated value or variations±0.5%, 1%, 5%, or 10% from a specified value.

The singular forms of the articles “a,” “an,” and “the” include plural references unless the context clearly dictates otherwise. For example, the term “a protein” or “at least one protein” can include a plurality of proteins, including mixtures thereof.

Compositions or methods “comprising” or “including” one or more recited elements may include other elements not specifically recited. For example, a composition that “comprises” or “includes” a protein may contain the protein alone or in combination with other ingredients.

As used herein, “about” means within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

### II. Compositions

### Engineered Antibody Fusion Proteins

In one embodiment, an isolated engineered antibody fusion protein with a redox-sensitive domain and modulable binding affinity for a target molecule is provided. In embodiments, the antibody fusion protein undergoes a conformational change upon exposure to an external signal and exhibits a different binding affinity to a target molecule.

As used herein, “isolated,” with respect to a polypeptide (and also a polynucleotide), means a molecule (e.g., polypeptide, protein or polynucleotide) isolated from its natural environment or prepared using synthetic methods such as those known to one of skill in the art. Complete purification is not required in either case. The molecules described herein can be isolated and purified from normally associated material in conventional ways, such that in the purified preparation the molecule is the predominant species in the preparation. At the very least, the degree of purification is such that extraneous material in the preparation does not interfere with use of the molecule in the manner disclosed herein. The molecule is at least about 85% pure; alternatively, at least about 90% pure, alternatively, at least about 95% pure; and alternatively, at least about 99% pure.

Disclosed herein are engineered antibody fusion proteins comprising a variable region of an immunoglobulin heavy chain (V_{H}), a variable region of an immunoglobulin light chain (V_{L}), and a redox-sensitive domain.

The V_{H} region and the V_{L} region are the variable regions of an immunoglobulin which are capable of binding to a target molecule.

The redox-sensitive domain (RSD) undergoes a conformational change upon exposure to an external signal. The redox-sensitive domain may be part of the Per-Arnt-Sim (PAS) superfamily of domains. The redox-sensitive domain may comprise a heme cofactor or a flavin cofactor. The flavin cofactor may be flavin mononucleotide (FMN). The redox-sensitive domain may a bacterium-derived cytochrome c-like molecule (CYTc) domain, a light-oxygen-voltage-sensing (LOV) domain, or a cGMP-specific phosphodiesterases, adenylyl cyclases, formate hydrogen lysate transcriptional activator (FhlA) (GAF) domain. In some embodiments, the redox-sensitive domain is a second LOV domain of a phototropin (LOV2). In some embodiments, the redox-sensitive domain is an LOV2 domain derived from Avena sativa (asLOV2).

The external signal may be redox potential or light. In some embodiments, the external signal is blue light. In some embodiments, the blue light has a wavelength of about 400-500 nm. In some embodiments, the blue light has a wavelength of about 450-490 nm, about 460-480 nm, about 465-475 nm, or about 470 nm. In other embodiments, the external signal is light with a wavelength between about 500-700 nm.

The V_{H} region and V_{L} region may be adjacent in the amino acid sequence of the antibody fusion protein. There may be a short peptide linker between the V_{H} region and the V_{L} region and the peptide linker may comprise glycine residues and/or serine residues. The linker may be between about 5 amino acids to about 30 amino acids in length. An amino acid sequence comprising a V_{H} region and a V_{L} region, optionally wherein a linker sequence is between the V_{H} region and the V_{L} region, may be referred to as single chain variable region (scFv) domain.

In some embodiments, the antibody fusion protein comprises a scFv domain (e.g., a V_{H} region and a V_{L} region optionally connected by a linker) and a redox-sensitive domain. In some embodiments, the redox-sensitive domain is located at the C-terminal end of the antibody fusion protein. In other embodiments, the redox-sensitive domain is located at the N-terminal end of the antibody fusion protein.

Peptides and proteins are said to have a C-terminal end (e.g., a C-terminus) and a N-terminal end (e.g., N-terminus). The C-terminus has a terminal amino acid residue with a free carboxylic acid (e.g., CO₂H) which is not bound to another amino acid residue as part of a peptide bond. The N-terminus has a free amino group (e.g., NH₂) which is not bound to another amino acid residue as part of a peptide bond.

In some embodiments, the redox-sensitive domain is located between the V_{H} region and the V_{L} region. In one embodiment, the VII region is located at the C-terminal end and the V_{L} region is located at the N-terminal end of the antibody fusion protein. In another embodiment, the V_{H} region is located at the N-terminal end and the V_{L} region is located at the C-terminal end of the antibody fusion protein.

The target molecule to which the V_{H} region and the V_{L} regions are capable of binding may be any biologically relevant molecule of interest. In embodiments, the target molecule is a metabolite, a hormone, and/or a therapeutic compound. Target molecules may be sugars, small molecules, peptides, or proteins. Target molecules may originate from any species of interest, including but not limited to proteins from human, monkey, mouse, rat, rabbit, dog, cat, horse, donkey, goat, pig, cow, bird, reptile, amphibian, fish, plant, fungi, bacteria, or Archean species. In some embodiments, the target molecule is insulin, glucagon, and glucagon-like peptide 1.

In an embodiment, the target molecule is insulin and the V_{H} region and the V_{L} region of the antibody fusion protein are anti-insulin. In some embodiments, the antibody fusion protein comprises an anti-human insulin scFv domain and/or an asLOV2 domain. In some embodiments, the antibody fusion protein has an scFv domain comprising an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 1. In one embodiment, the antibody fusion protein comprises the amino acid sequence set forth in SEQ ID NO: 1.

Anti-human insulin scFv (Clone HB125)
(SEQ ID NO: 1)
MAQIQLVQSGPELKKPGETVKISCKASGYTFTDYSMHWVKQAPGKGLKW
MDWINTETGVPTYADDFKGRFAFSLETSANTAYLQINDLKNEDTATYFC
TRGYGKGYFDVWGAGTTVTVSSAKSTPPSVYPLAPGSGGGGSGGGGSGG
GGSQIVLTQSPTIMSASLGERVTMTCTASSSVSSSYLHWYQQKPGSSPK
LWIYSTSNLASGVPARFSGSGSGTSYSLTISSMEAEDAATYYCHQYHRS
PPTFGAGTKLELKRADAA.

In some embodiments, the antibody fusion protein comprises an LOV2 domain from Avena sativa (asLOV2) as the redox sensitive domain. In some embodiments, the antibody fusion protein has a redox-sensitive domain comprising an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 2. In one embodiment, the antibody fusion protein comprises the amino acid sequence set forth in SEQ ID NO: 2.

LOV2 derived from Avena sativa (asLOV2)
(SEQ ID NO: 2)
GEFLATTLERIEKNFVITDPRLPDNPIIFASDSFLQLTEYSREEILGRN
CRFLQGPETDRATVRKIRDAIDNQTEVTVQLINYTKSGKKFWNLFHLQP
MRDQKGDVQYFIGVQLDGTEHVRDAAEREAVMLIKKTAEEIDEAAKEL

In some embodiments, the antibody fusion protein comprises a truncated LOV2 domain from Avena sativa as the redox sensitive domain (tLOV2). In some embodiments, the antibody fusion protein has a redox-sensitive domain comprising an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 36. In one embodiment, the antibody fusion protein comprises the amino acid sequence set forth in SEQ ID NO: 36.

truncated LOV2 derived from Avena sativa (tLOV2)
(SEQ ID NO: 36)
GEFLATTLERIEKNFVITDPRLPDNPIIFASDSFLQLTEYSREEILGRN
CRFLQGPETDRATVRKIRDAIDNQTEVTVQLINYTKSGKKFWNLFHLQP
MRDQKGDVQYFIGVQLDGTEHVRDAAEREAVMLIKKTA

In some embodiments the antibody fusion protein comprises an anti-human insulin scFv domain and an asLOV2 domain. In one embodiment, the asLOV2 domain is at the C-terminal end. In one embodiment, the antibody fusion protein comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 3 or SEQ ID NO: 16. In one embodiment, the antibody fusion protein comprises an amino acid sequence set forth in SEQ ID NO: 3 or SEQ ID NO: 16.

Anti-human insulin scFV fusion with asLOV2 at C-
terminus (cLOV)
(SEQ ID NO: 3)
MAQIQLVQSGPELKKPGETVKISCKASGYTFTDYSMHWVKQAPGKGLKW
MDWINTETGVPTYADDFKGRFAFSLETSANTAYLQINDLKNEDTATYFC
TRGYGKGYFDVWGAGTTVTVSSAKSTPPSVYPLAPGSGGGGSGGGGSGG
GGSQIVLTQSPTIMSASLGERVTMTCTASSSVSSSYLHWYQQKPGSSPK
LWIYSTSNLASGVPARFSGSGSGTSYSLTISSMEAEDAATYYCHQYHRS
PPTFGAGTKLELKRADAAGGGGSGEFLATTLERIEKNFVITDPRLPDNP
IIFASDSFLQLTEYSREEILGRNCRFLQGPETDRATVRKIRDAIDNQTE
VTVQLINYTKSGKKFWNLFHLQPMRDQKGDVQYFIGVQLDGTEHVRDAA
EREAVMLIKKTAEEIDEAAKEL;
or
(SEQ ID NO: 16)
MAQIQLVQSGPELKKPGETVKISCKASGYTFTDYSMHWVKQAPGKGLKW
MDWINTETGVPTYADDFKGFAFSLETSANTAYLQINDLKNEDTATYFCT
RGYGKGYFDVWGAGTTVTVSSAKSTPPSVYPLAPGSGGGGSGGGGSGGG
GSQIVLTQSPTIMSASLGERVTTCTASSSVSSSYLHWYQQKPGSSPKLW
IYSTSNLASGVPARFSGSGSGTSYSLTISSMEAEDAATYYCHQYHRSPP
TFGAGTKLELKRADAAGGGGGEFLATTLERIEKNFVITDPRLPDNPIIF
ASDSFLQLTEYSREEILGRNCRFLQGPETDRATVRKIRDAIDNQTEVTV
QLINYTKSGKKFWNLFHLQPMRDQKGQIGVQLDGTEHVRDAAEREAVML
IKKTAEEIDEAAKELKL

In another embodiment, the antibody fusion protein comprises an anti-human insulin scFv domain and an asLOV2 domain, wherein the asLOV2 domain is at the N-terminal end. In one embodiment, the antibody fusion protein comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 4 or SEQ ID NO: 17. In one embodiment, the antibody fusion protein comprises an amino acid sequence set forth in SEQ ID NO: 4 or SEQ ID NO: 17.

Anti-human insulin scFV fusion with asLOV2 at N-
terminus (nLOV)
(SEQ ID NO: 4)
MGEFLATTLERIEKNFVITDPRLPDNPIIFASDSFLQLTEYSREEILGR
NCRFLQGPETDRATVRKIRDAIDNQTEVTVQLINYTKSGKKFWNLFHLQ
PMRDQKGDVQYFIGVQLDGTEHVRDAAEREAVMLIKKTAEEIDEAAGGG
GSAQIQLVQSGPELKKPGETVKISCKASGYTFTDYSMHWVKQAPGKGLK
WMDWINTETGVPTYADDFKGRFAFSLETSANTAYLQINDLKNEDTATYF
CTRGYGKGYFDVWGAGTTVTVSSAKSTPPSVYPLAPGSGGGGSGGGGSG
GGGSQIVLTQSPTIMSASLGERVTMTCTASSSVSSSYLHWYQQKPGSSP
KLWIYSTSNLASGVPARFSGSGSGTSYSLTISSMEAEDAATYYCHQYHR
SPPTFGAGTKLELKRADAA;
or
(SEQ ID NO: 17)
MGEFLATTLERIEKNFVITDPRLPDNPIIFASDSFLQLTEYSREEILGR
NCRFLQGPETDRATVRKIRDAIDNQTEVTVQLINYTKSGKKFWNLFHLQ
PMRDQKGDVQYFIGVQLDGTEHVRDAAEREAVMLIKKTAEEIDEAAKEL
AQIQLVQSGPELKKPGETVKISCKASGYTFTDYSMHWVKQAPGKGLKWM
DWINTETGVPTYADDFKGRFAFSLETSANTAYLQINDLKNEDTATYFCT
RGYGKGYFDVWGAGTTVTVSSAKSTPPSVYPLAPGSGGGGSGGGGSGGG
GSQIVLTQSPTIMSASLGERVTMTCTASSSVSSSYLHWYQQKPGSSPKL
WIYSTSNLASGVPARFSGSGSGTSYSLTISSMEAEDAATYYCHQYHRSP
PTFGAGTKLELKRADAAKL

In some embodiments, the antibody fusion protein comprises a truncated asLOV2 domain at the N-terminus. In one embodiment, the antibody fusion protein has an amino acid sequence comprising at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 18. In one embodiment, the antibody fusion protein comprises an amino acid sequence set forth in SEQ ID NO: 18.

Anti-human insulin scFV fusion with truncated
asLOV2 at N-terminus (ntLOV)
(SEQ ID NO: 18)
MGEFLATTLERIEKNFVITDPRLPDNPIIFASDSFLQLTEYSREEILGR
NCRFLQGPETDRATVRKIRDAIDNQTEVTVQLINYTKSGKKFWNLFHLQ
PMRDQKGDVQYFIGVQLDGTEHVRDAAEREAVMLIKKTAAQIQLVQSGP
ELKKPGETVKISCKASGYTFTDYSMHWVKQAPGKGLKWMDWINTETGVP
TYADDFKGRFAFSLETSANTAYLQINDLKNEDTATYFCTRGYGKGYFDV
WGAGTTVTVSSAKSTPPSVYPLAPGSGGGGSGGGGSGGGGSQIVLTQSP
TIMSASLGERVTMTCTASSSVSSSYLHWYQQKPGSSPKLWIYSTSNLAS
GVPARFSGSGSGTSYSLTISSMEAEDAATYYCHQYHRSPPTFGAGTKLE
LKRADAAKL

In some embodiments, the antibody fusion protein comprises a V_{H} region and a V_{L} region, which are not directly adjacent to each other. In some embodiments, the redox-sensitive domain is between the V_{H} region and a V_{L} region. In one embodiment, the V_{H} region and the V_{L} region are anti-human insulin. In some embodiments, the VII region comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 6. In one embodiment, the V_{H} region comprises an amino acid sequence set forth in SEQ ID NO: 6.

V_{H} region of anti-human insulin
(SEQ ID NO: 6)
AQIQLVQSGPELKKPGETVKISCKASGYTFTDYSMHWVKQAPGKGLKWM
DWINTETGVPTYADDFKGRFAFSLETSANTAYLQINDLKNEDTATYFCT
RGYGKGYFDVWGAGTTVTVSSAKSTPPSVYPLAPGS

In some embodiments, the V_{L} region comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 7. In one embodiment, the V_{H} region comprises an amino acid sequence set forth in SEQ ID NO: 7.

V_{L} region of anti-human insulin
(SEQ ID NO: 7)
QIVLTQSPTIMSASLGERVTMTCTASSSVSSSYLHWYQQKPGSSPKLWI
YSTSNLASGVPARFSGSGSGTSYSLTISSMEAEDAATYYCHQYHRSPPT
FGAGTKLELKRADAA

In some embodiments, the antibody fusion protein has an amino acid sequence comprising: i) a V_{H} region with at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 6; ii) a V_{L} region with at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 7; and iii) an asLOV2 redox-sensitive domain with at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO:2 or SEQ ID NO: 36. In one embodiment, the antibody fusion protein has an amino acid sequence comprising: i) a V_{H} region comprising the amino acid sequence set forth in SEQ ID NO: 6; ii) a V_{H} region comprising the amino acid sequence set forth in SEQ ID NO: 7; and iii) an asLOV2 domain comprising the amino acid sequence set forth in SEQ ID NO: 2 or SEQ ID NO: 36.

In one embodiment, the antibody fusion protein has an amino acid sequence comprising at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 5 or SEQ ID NO: 19. In one embodiment, the antibody fusion protein comprises an amino acid sequence set forth in SEQ ID NO: 5 or SEQ ID NO: 19.

Anti-human insulin V_{H}-asLOV2-V_{L} with glycine-rich
linkers (gsLOV)
(SEQ ID NO: 5)
MAQIQLVQSGPELKKPGETVKISCKASGYTFTDYSMHWVKQAPGKGLKW
MDWINTETGVPTYADDFKGRFAFSLETSANTAYLQINDLKNEDTATYFC
TRGYGKGYFDVWGAGTTVTVSSAKSTPPSVYPLAPGSGGGGSGEFLATT
LERIEKNFVITDPRLPDNPIIFASDSFLQLTEYSREEILGRNCRFLQGP
ETDRATVRKIRDAIDNQTEVTVQLINYTKSGKKFWNLFHLQPMRDQKGD
VQYFIGVQLDGTEHVRDAAEREAVMLIKKTAEEIDEAAGGGGSQIVLTQ
SPTIMSASLGERVTMTCTASSSVSSSYLHWYQQKPGSSPKLWIYSTSNL
ASGVPARFSGSGSGTSYSLTISSMEAEDAATYYCHQYHRSPPTFGAGTK
LELKRADAA;
(SEQ ID NO: 19)
MAQIQLVQSGPELKKPGETVKISCKASGYTFTDYSMHWVKQAPGKGLKW
MDWINTETGVPTYADDFKGRFAFSLETSANTAYLQINDLKNEDTATYFC
TRGYGKGYFDVWGAGTTVTVSSAKSTPPSVYPLAPGSGGGGSGEFLATT
LERIEKNFVITDPRLPDNPIIFASDSFLQLTEYSREEILGRNCRFLQGP
ETDRATVRKIRDAIDNQTEVTVQLINYTKSGKKFWNLFHLQPMRDQKGD
VQYFIGVQLDGTEHVRDAAEREAVMLIKKTAEEIDEAAKELGGGGSQIV
LTQSPTIMSASLGERVTMTCTASSSVSSSYLHWYQQKPGSSPKLWIYST
SNLASGVPARFSGSGSGTSYSLTISSMEAEDAATYYCHQYHRSPPTFGA
GTKLELKRADAAKL.

In one embodiment, the antibody fusion protein has an amino acid sequence comprising at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 20. In one embodiment, the antibody fusion protein comprises an amino acid sequence set forth in SEQ ID NO: 20.

Anti-human insulin V_{H}-asLOV2-V_{L} without glycine-
rich linkers (nolinkLOV)
(SEQ ID NO: 20)
MAQIQLVQSGPELKKPGETVKISCKASGYTFTDYSMHWVKQAPGKGLKW
MDWINTETGVPTYADDFKGRFAFSLETSANTAYLQINDLKNEDTATYFC
TRGYGKGYFDVWGAGTTVTVSSAKSTPPSVYPLAPGSGEFLATTLERIE
KNFVITDPRLPDNPIIFASDSFLQLTEYSREEILGRNCRFLQGPETDRA
TVRKIRDAIDNQTEVTVQLINYTKSGKKFWNLFHLQPMRDQKGDVQYFI
GVQLDGTEHVRDAAEREAVMLIKKTAEEIDEAAKELQIVLTQSPTIMSA
SLGERVTMTCTASSSVSSSYLHWYQQKPGSSPKLWIYSTSNLASGVPAR
FSGSGSGTSYSLTISSMEAEDAATYYCHQYHRSPPTFGAGTKLELKRAD
AAKL

Variants of antibody fusion proteins can be prepared to fix the proteins in a “dark state” or a “light state” by mutating residues of the redox-sensitive domain (e.g., LOV2). These variants can be used in order to test and compare the binding affinities of the antibody fusion protein in the different states. Provided are amino acid sequences for various antibody fusion proteins fixed in light and dark states.

Anti-human insulin scFV fusion with asLOV2 at C-terminus (cLOV) fixed in
“dark state” (mutant of SEQ ID NO: 3)
(SEQ ID NO: 21)
MAQIQLVQSGPELKKPGETVKISCKASGYTFTDYSMHWVKQAPGKGLKWMDWI
NTETGVPTYADDFKGRFAFSLETSANTAYLQINDLKNEDTATYFCTRGYGKGYF
DVWGAGTTVTVSSAKSTPPSVYPLAPGSGGGGSGGGGSGGGGSQIVLTQSPTIMS
ASLGERVTMTCTASSSVSSSYLHWYQKPGSSPKLWIYSTSNLASGVPARFSGSGS
GTSYSLTISSMEAEDAATYYCHQYHRSPPTFGAGTKLELKRADAAGGGGSGEFL
ATLERIEKNFVITDPRLPDNPIIFASDSFLQLTEYSREEILGRNARFLQGPETDRATV
RKIRDAIDNQTEVTVQLINYTKSGKKFWNLFHLQPMRDQKGDVQYFIGVQLDGT
EHVRDAAEREAVMLIKKTAEEIDEAAKELKL
Anti-human insulin scFV fusion with asLOV2 at C-terminus (cLOV) fixed in
“light state” (mutant of SEQ ID NO: 3)
(SEQ ID NO: 22)
MAQIQLVQSGPELKKPGETVKISCKASGYTFTDYSMHWVKQAPGKGLKWMDWI
NTETGVPTYADDFKGRFAFSLETSANTAYLQINDLKNEDTATYFCTRGYGKGYF
DVWGAGTTVTVSSAKSTPPSVYPLAPGSGGGGSGGGGSGGGGSQIVLTQSPTIMS
ASLGERVTMTCTASSSVSSSYLHWYQQKPGSSPKLWIYSTSNLASGVPARFSGSG
SGTSYSLTISSMEAEDAATYYCHQYHRSPPTFGAGTKLELKRADAAGGGGSGEF
LATTLERIEKNFVITDPRLPDNPIIFASDSFLQLTEYSREEILGRNCRFLQGPETDRA
TVRKIRDAIDNQTEVTVQLINYTKSGKKFWNLFHLQPMRDQKGDVQYFIGVQLD
GTEHVRDAAEREAVMLEKKTEEEIDEAAKELKL
Anti-human insulin scFV fusion with asLOV2 at N-terminus (nLOV) fixed in
a “dark state” (mutant of SEQ ID NO: 17)
(SEQ ID NO: 23)
MGEFLATTLERIEKNFVITDPRLPDNPIIFASDSFLQLTEYSREEILGRNARFLQGPE
TDRATVRKIRDAIDNQTEVTVQLINYTKSGKKFWNLFHLQPMRDQKGDVQYFIG
VQLDGTEHVRDAAEREAVMLIKKTAEEIDEAAKELAQIQLVQSGPELKKPGETV
KISCKASGYTFTDYSMHWVKQAPGKGLKWMDWINTETGVPTYADDFKGRFAFS
LETSANTAYLQINDLKNEDTATYFCTRGYGKGYFDVWGAGTTVTVSSAKSTPPS
VYPLAPGSGGGGSGGGGSGGGGSQIVLTQSPTIMSASLGERVTMTCTASSSVSSS
YLHWYQQKPGSSPKLWIYSTSNLASGVPARFSGSGSGTSYSLTISSMEAEDAATY
YCHQYHRSPPTFGAGTKLELKRADAAKL
Anti-human insulin scFV fusion with asLOV2 at N-terminus (nLOV) fixed in
a “light state” (mutant of SEQ ID NO: 17)
(SEQ ID NO: 24)
MGEFLATTLERIEKNFVITDPRLPDNPIIFASDSFLQLTEYSREEILGRNCRFLQGPE
TDRATVRKIRDAIDNQTEVTVQLINYTKSGKKFWNLFHLQPMRDQKGDVQYFIG
VQLDGTEHVRDAAEREAVMLEKKTEEEIDEAAKELAQIQLVQSGPELKKPGETV
KISCKASGYTFTDYSMHWVKQAPGKGLKWMDWINTETGVPTYADDFKGRFAFS
LETSANTAYLQINDLKNEDTATYFCTRGYGKGYFDVWGAGTTVTVSSAKSTPPS
VYPLAPGSGGGGSGGGGSGGGGSQIVLTQSPTIMSASLGERVTMTCTASSSVSSS
YLHWYQQKPGSSPKLWIYSTSNLASGVPARFSGSGSGTSYSLTISSMEAEDAATY
YCHQYHRSPPTFGAGTKLELKRADAAKL
Anti-human insulin scFV fusion with truncated asLOV2 at N-terminus (ntLOV)
fixed in a “dark state” (mutant of SEQ ID NO: 18)
(SEQ ID NO: 25)
MGEFLATTLERIEKNFVITDPRLPDNPIIFASDSFLQLTEYSREEILGRNARFLQGPE
TDRATVRKIRDAIDNQTETVQLINYTKSGKKFWNLFHLQPMRDQKGDVQYFIGV
QLDGTEHVRDAAEREAVMLIKKTAAQIQLVQSGPELKKPGETVKISCKASGYTFT
DYSMHWVKQAPGKGLKWMDWINTETGVPTYADDFKGRFAFSLETSANTAYLQI
NDLKNEDTATYFCTRGYGKGYFDVWGAGTTVTVSSAKSTPPSVYPLAPGSGGG
GSGGGGSGGGGSQIVLTSPTIMSASLGERVTMTCTASSSVSSSYLHWYQQKPGSS
PKLWIYSTSNLASGVPARFSGSGSGTSYSLTISSMEAEDAATYYCHQYHRSPPTFG
AGTKLELKRADAAKL
Anti-human insulin scFV fusion with truncated asLOV2 at N-terminus (ntLOV)
fixed in a “light state” (mutant of SEQ ID NO: 18)
(SEQ ID NO: 26)
MGEFLATTLERIEKNFVITDPRLPDNPIIFASDSFLQLTEYSREEILGRNCRFLQGPE
TDRATVRKIRDAIDNQTEVTVQLINYTKSGKKFWNLFHLQPMRDQKGDVQYFIG
VQLDGTEHVRDAAEREAVMLEKKTEAQIQLVQSGPELKKPGETVKISCKASGYT
FTDYSMHWVKQAPGKGLKWMDWINTETGVPTYADDFKGRFAFSLETSANTAY
LQINDLKNEDTATYFCTRGYGKGYFDVWGAGTTVTVSSAKSTPPSVYPLAPGSG
GGGSGGGGSGGGGSQIVLTQSPTIMSASLGERVTMTCTASSSVSSSYLHWYQQK
PGSSPKLWIYSTSNLASGVPARFSGSGSGTSYSLTISSMEAEDAATYYCHQYHRSP
PTFGAGTKLELKRADAAKL
Anti-human insulin V_{H}-asLOV2-V_{L} with glycine-rich linkers (gsLOV) fixed in
a “dark state” (mutant of SEQ ID NO: 19)
(SEQ ID NO: 27)
MAQIQLVQSGPELKKPGETVKISCKASGYTFTDYSMHWVKQAPGKGLKWMDWI
NTETGVPTYADDFKGRFAFSLETSANTAYLQINDLKNEDTATYFCTRGYGKGYF
DVWGAGTTVTVSSAKSTPPSVYPLAPGSGGGGSGEFLATTLERIEKNFVITDPRLP
DNPIIFASDSFLQLTEYSREEILGRNARFLQGPETDRATVRKIRDAIDNQTEVTVQL
INYTKSGKKFWNLFHLQPMRDQKGDVQYFIGVQLDGTEHVRDAAEREAVMLIK
KTAEEIDEAAKELGGGGSQIVLTQSPTIMSASLGERVTMTCTASSSVSSSYLHWY
QQKPGSSPKLWIYSTSNLASGVPARFSGSGSGTSYSLTISSMEAEDAATYYCHQY
HRSPPTFGAGTKLELKRADAAKL
Anti-human insulin V_{H}-asLOV2-V_{L} with glycine-rich linkers (gsLOV) fixed in
a “light state” (mutant of SEQ ID NO: 19)
(SEQ ID NO: 28)
MAQIQLVQSGPELKKPGETVKISCKASGYTFTDYSMHWVKQAPGKGLKWMDWI
NTETGVPTYADDFKGRFAFSLETSANTAYLQINDLKNEDTATYFCTRGYGKGYF
DVWGAGTTVTVSSAKSTPPSVYPLAPGSGGGGSGEFLATTLERIEKNFVITDPRLP
DNPIIFASDSFLQLTEYSREEILGRNCRFLQGPETDRATVRKIRDAIDNQTEVTVQL
INYTKSGKKFWNLFHLQPMRDQKGDVQYFIGVQLDGTEHVRDAAEREAVMLEK
KTEEEIDEAAKELGGGGSQIVLTQSPTIMSASLGERVTMTCTASSSVSSSYLHWY
QQKPGSSPKLWIYSTSNLASGVPARFSGSGSGTSYSLTISSMEAEDAATYYCHQY
HRSPPTFGAGTKLELKRADAAKL
Anti-human insulin V_{H}-asLOV2-V_{L} without glycine-rich linkers (nolinkLOV)
fixed in a “dark state” (mutant of SEQ ID NO: 20)
(SEQ ID NO: 29)
MAQIQLVQSGPELKKPGETVKISCKASGYTFTDYSMHWVKQAPGKGLKWMDWI
NTETGVPTYADDFKGRFAFSLETSANTAYLQINDLKNEDTATYFCTRGYGKGYF
DVWGAGTTVTVSSAKSTPPSVYPLAPGSGEFLATTLERIEKNFVITDPRLPDNPIIF
ASDSFLQLTEYSREEILGRNARFLQGPETDRATVRKIRDAIDNQTEVTVQLINYTK
SGKKFWNLFHLQPMRDQKGDVQYFIGVQLDGTEHVRDAAEREAVMLIKKTAEE
IDEAAKELQIVLTQSPTIMSASLGERVTMTCTASSSVSSSYLHWYQQKPGSSPKL
WIYSTSNLASGVPARFSGSGSGTSYSLTISSMEAEDAATYYCHQYHRSPPTFGAG
TKLELKRADAAKL
Anti-human insulin V_{H}-asLOV2-V_{L} without glycine-rich linkers (nolinkLOV)
fixed in a “light state” (mutant of SEQ ID NO: 20)
(SEQ ID NO: 30)
MAQIQLVQSGPELKKPGETVKISCKASGYTFTDYSMHWVKQAPGKGLKWMDWI
NTETGVPTYADDFKGRFAFSLETSANTAYLQINDLKNEDTATYFCTRGYGKGYF
DVWGAGTTVTVSSAKSTPPSVYPLAPGSGEFLATTLERIEKNFVITDPRLPDNPIIF
ASDSFLQLTEYSREEILGRNCRFLQGPETDRATVRKIRDAIDNQTEVTVQLINYTK
SGKKFWNLFHLQPMRDQKGDVQYFIGVQLDGTEHVRDAAEREAVMLEKKTEEE
IDEAAKELQIVLTQSPTIMSASLGERVTMTCTASSSVSSSYLHWYQQKPGSSPKL
WIYSTSNLASGVPARFSGSGSGTSYSLTISSMEAEDAATYYCHQYHRSPPTFGAG
TKLELKRADAAKL

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of nonconservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

It may be desired with respect to biosensors to engineer an antibody capable of modulating its binding affinity to its target molecule (e.g., antigen) based on physicochemical conditions (e.g., irradiation with light) to be able to regenerate the antigen-binding sites of the antibody in situ for continuous monitoring applications. The antibody fusion proteins disclosed herein have a binding affinity towards a target molecule which is modulated upon exposure to an external signal (e.g., redox potential or light).

In some embodiments, a sensitivity of the antibody fusion protein towards its target molecule is modulated upon exposure to the external signal. In some embodiments, the sensitivity of the antibody fusion protein towards the target molecule is at least 2-fold, at least 5-fold, at least 10-fold, at least 100-fold, or at least 1000-fold different upon exposure to the external signal. In some embodiments, a limit of detection (LOD) of the antibody fusion protein is modulated upon exposure to the external signal. In some embodiments, a specific binding ability of the antibody fusion protein towards its target molecule is modulated by exposure to the external signal. In some embodiments, the target molecule is at least 2-fold, at least 5-fold, at least 10-fold, at least 100-fold, or at least 1000-fold different upon exposure to the external signal. In some embodiments, the dissociation constant (K_{d}) of the antibody fusion protein towards the target molecule is modulated by exposure to the external signal. In some embodiments, the K_{d} of the antibody fusion protein is at least 10, at least 10², at least 10³, at least 10⁴, at least 10⁵, at least 10⁶, at least 10⁷, at least 10⁸, at least 10⁹, or at least 10¹⁰ times different upon exposure to the external signal.

### Polynucleotides Encoding Antibody Fusion Proteins

In one embodiment, an isolated polynucleotide that encodes for an engineered antibody fusion protein is described herein. In some embodiments, the polynucleotide comprises a nucleotide sequence encoding an amino acid sequence comprising any one of SEQ ID NO: 3-5, and 16-20.

The terms “nucleic acid” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides 5 in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

In some embodiments, the polynucleotide encoding the antibody fusion protein comprises a nucleotide sequence having at least 90%, at least 91%, at least 92%, at least 93%, at least 94%, at least 95%, at least 96%, at least 97%, at least 98%, at least 99%, at least 99.5%, or at least 99.9% sequence identity to any one of SEQ ID NOs: 8-10, and 21-25. In some embodiments, the polynucleotide encoding the antibody fusion protein comprises a nucleotide sequence set forth in any one of SEQ ID NOs: 8-10, and 21-25.

Anti-human insulin fusion with scFV-asLOV2 at C-terminus (cLOV)
(SEQ ID NO: 8)
ATGGCACAGATCCAGTTGGTGCAGTCTGGACCTGAACTGAAGAAGCCTGGAG
AGACAGTCAAGATCTCCTGCAAGGCTTCTGGTTATACCTTCACAGACTATTCA
ATGCACTGGGTGAAGCAGGCTCCAGGAAAGGGTTTAAAGTGGATGGACTGG
ATAAACACTGAGACTGGTGTGCCAACCTATGCAGATGACTTCAAGGGACGGT
TTGCCTTCTCTTTGGAAACCTCTGCTAACACTGCCTATTTGCAGATCAACGAC
CTCAAAAATGAGGACACGGCTACATATTTCTGTACTAGAGGGTATGGAAAGG
GGTACTTCGATGTCTGGGGCGCAGGGACCACGGTCACCGTCTCCTCAGCCAA
AAGCACACCCCCATCCGTTTATCCATTGGCCCCTGGAAGCGGCGGAGGCGGA
TCAGGAGGAGGAGGATCAGGCGGAGGAGGATCACAAATTGTTCTCACCCAGT
CTCCAACAATCATGTCTGCATCTCTAGGGGAACGGGTCACCATGACCTGCAC
TGCCAGCTCAAGTGTAAGTTCCAGTTACTTGCACTGGTACCAGCAGAAGCCA
GGATCCTCCCCCAAACTCTGGATTTATAGTACATCCAACCTGGCTTCTGGAGT
CCCAGCTCGCTTCAGTGGCAGTGGGTCTGGGACCTCTTACTCTCTCACAATCA
GCAGCATGGAGGCTGAAGATGCTGCCACTTATTACTGCCACCAGTATCATCG
TTCCCCACCCACGTTCGGTGCTGGGACCAAGCTGGAGCTGAAACGGGCTGAT
GCTGCAGGCGGAGGCGGATCAGGCGAATTTCTGGCGACCACCCTGGAACGCA
TTGAAAAAAACTTTGTGATTACCGATCCGCGCCTGCCGGATAACCCGATTATT
TTTGCGAGCGATAGCTTTCTGCAGCTGACCGAATATAGCCGCGAAGAAATTC
TGGGCCGCAACTGCCGCTTTCTGCAGGGCCCGGAAACCGATCGCGCGACCGT
GCGCAAAATTCGCGATGCGATTGATAACCAG;
or
(SEQ ID NO: 31; encoding SEQ ID NO: 16)
CATATGGCACAGATCCAGTTGGTGCAGTCTGGACCTGAACTGAAGAAGCCTG
GAGAGACAGTCAAGATCTCCTGCAAGGCTTCTGGTTATACCTTCACAGACTAT
TCAATGCACTGGGTGAAGCAGGCTCCAGGAAAGGGTTTAAAGTGGATGGACT
GGATAAACACTGAGACTGGTGTGCCAACCTATGCAGATGACTTCAAGGGACG
GTTTGCCTTCTCTTTGGAAACCTCTGCTAACACTGCCTATTTGCAGATCAACG
ACCTCAAAAATGAGGACACGGCTACATATTTCTGTACTAGAGGGTATGGAAA
GGGGTACTTCGATGTCTGGGGCGCAGGGACCACGGTCACCGTCTCCTCAGCC
AAAAGCACACCCCCATCCGTTTATCCATTGGCCCCTGGAAGCGGCGGAGGCG
GATCAGGAGGAGGAGGATCAGGCGGAGGAGGATCACAAATTGTTCTCACCC
AGTCTCCAACAATCATGTCTGCATCTCTAGGGGAACGGGTCACCATGACCTG
CACTGCCAGCTCAAGTGTAAGTTCCAGTTACTTGCACTGGTACCAGCAGAAG
CCAGGATCCTCCCCCAAACTCTGGATTTATAGTACATCCAACCTGGCTTCTGG
AGTCCCAGCTCGCTTCAGTGGCAGTGGGTCTGGGACCTCTTACTCTCTCACAA
TCAGCAGCATGGAGGCTGAAGATGCTGCCACTTATTACTGCCACCAGTATCA
TCGTTCCCCACCCACGTTCGGTGCTGGGACCAAGCTGGAGCTGAAACGGGCT
GATGCTGCAGGCGGAGGCGGATCAGGCGAATTTCTGGCGACCACCCTGGAAC
GCATTGAAAAAAACTTTGTGATTACCGATCCGCGCCTGCCGGATAACCCGAT
TATTTTTGCGAGCGATAGCTTTCTGCAGCTGACCGAATATAGCCGCGAAGAA
ATTCTGGGCCGCAACTGCCGCTTTCTGCAGGGCCCGGAAACCGATCGCGCGA
CCGTGCGCAAAATTCGCGATGCGATTGATAACCAGACCGAAGTGACCGTGCA
GCTGATTAACTATACCAAAAGCGGCAAAAAATTTTGGAACCTGTTTCATCTGC
AGCCGATGCGCGATCAGAAAGGCGATGTGCAGTATTTTATTGGCGTGCAGCT
GGATGGCACCGAACATGTGCGCGATGCGGCGGAACGCGAAGCGGTGATGCT
GATTAAAAAAACCGCGGAAGAAATTGATGAAGCGGCGAAAGAACTGAAGCT
T.
Anti-human insulin scFV fusion with asLOV2 at N-terminus (nLOV)
(SEQ ID NO: 9)
ATGGGCGAATTTCTGGCGACCACCCTGGAACGCATTGAAAAAAACTTTGTGA
TTACCGATCCGCGCCTGCCGGATAACCCGATTATTTTTGCGAGCGATAGCTTT
CTGCAGCTGACCGAATATAGCCGCGAAGAAATTCTGGGCCGCAACTGCCGCT
TTCTGCAGGGCCCGGAAACCGATCGCGCGACCGTGCGCAAAATTCGCGATGC
GATTGATAACCAGACCGAAGTGACCGTGCAGCTGATTAACTATACCAAAAGC
GGCAAAAAATTTTGGAACCTGTTTCATCTGCAGCCGATGCGCGATCAGAAAG
GCGATGTGCAGTATTTTATTGGCGTGCAGCTGGATGGCACCGAACATGTGCG
CGATGCGGCGGAACGCGAAGCGGTGATGCTGATTAAAAAAACCGCGGAAGA
AATTGATGAAGCGGCGAAAGAACTGGGTAGTGGTGCACAGATCCAGTTGGTG
CAGTCTGGACCTGAACTGAAGAAGCCTGGAGAGACAGTCAAGATCTCCTGCA
AGGCTTCTGGTTATACCTTCACAGACTATTCAATGCACTGGGTGAAGCAGGCT
CCAGGAAAGGGTTTAAAGTGGATGGACTGGATAAACACTGAGACTGGTGTGC
CAACCTATGCAGATGACTTCAAGGGACGGTTTGCCTTCTCTTTGGAAACCTCT
GCTAACACTGCCTATTTGCAGATCAACGACCTCAAAAATGAGGACACGGCTA
CATATTTCTGTACTAGAGGGTATGGAAAGGGGTACTTCGATGTCTGGGGCGC
AGGGACCACGGTCACCGTCTCCTCAGCCAAAAGCACACCCCCATCCGTTTAT
CCATTGGCCCCTGGAAGCGGCGGAGGCGGATCAGGAGGAGGAGGATCAGGC
GGAGGAGGATCACAAATTGTTCTCACCCAGTCTCCAACAATCATGTCTGCATC
TCTAGGGGAACGGGTCACCATGACCTGCACTGCCAGCTCAAGTGTAAGTTCC
AGTTACTTGCACTGGTACCAGCAGAAGCCAGGATCCTCCCCCAAACTCTGGA
TTTATAGTACATCCAACCTGGCTTCTGGAGTCCCAGCTCGCTTCAGTGGCAGT
GGGTCTGGGACCTCTTACTCTCTCACAATCAGCAGCATGGAGGCTGAAGATG
CTGCCACTTATTACTGCCACCAGTATCATCGTTCCCCACCCACGTTCGGTGCT
GGGACCAAGCTGGAGCTGAAACGGGCTGATGCTGC;
or
(SEQ ID NO: 32; encoding SEQ ID NO: 17)
CATATGGGCGAATTTCTGGCGACCACCCTGGAACGCATTGAAAAAAACTTTG
TGATTACCGATCCGCGCCTGCCGGATAACCCGATTATTTTTGCGAGCGATAGC
TTTCTGCAGCTGACCGAATATAGCCGCGAAGAAATTCTGGGCCGCAACTGCC
GCTTTCTGCAGGGCCCGGAAACCGATCGCGCGACCGTGCGCAAAATTCGCGA
TGCGATTGATAACCAGACCGAAGTGACCGTGCAGCTGATTAACTATACCAAA
AGCGGCAAAAAATTTTGGAACCTGTTTCATCTGCAGCCGATGCGCGATCAGA
AAGGCGATGTGCAGTATTTTATTGGCGTGCAGCTGGATGGCACCGAACATGT
GCGCGATGCGGCGGAACGCGAAGCGGTGATGCTGATTAAAAAAACCGCGGA
AGAAATTGATGAAGCGGCGAAAGAACTGGCACAGATCCAGTTGGTGCAGTCT
GGACCTGAACTGAAGAAGCCTGGAGAGACAGTCAAGATCTCCTGCAAGGCTT
CTGGTTATACCTTCACAGACTATTCAATGCACTGGGTGAAGCAGGCTCCAGG
AAAGGGTTTAAAGTGGATGGACTGGATAAACACTGAGACTGGTGTGCCAACC
TATGCAGATGACTTCAAGGGACGGTTTGCCTTCTCTTTGGAAACCTCTGCTAA
CACTGCCTATTTGCAGATCAACGACCTCAAAAATGAGGACACGGCTACATAT
TTCTGTACTAGAGGGTATGGAAAGGGGTACTTCGATGTCTGGGGCGCAGGGA
CCACGGTCACCGTCTCCTCAGCCAAAAGCACACCCCCATCCGTTTATCCATTG
GCCCCTGGAAGCGGCGGAGGCGGATCAGGAGGAGGAGGATCAGGCGGAGGA
GGATCACAAATTGTTCTCACCCAGTCTCCAACAATCATGTCTGCATCTCTAGG
GGAACGGGTCACCATGACCTGCACTGCCAGCTCAAGTGTAAGTTCCAGTTAC
TTGCACTGGTACCAGCAGAAGCCAGGATCCTCCCCCAAACTCTGGATTTATA
GTACATCCAACCTGGCTTCTGGAGTCCCAGCTCGCTTCAGTGGCAGTGGGTCT
GGGACCTCTTACTCTCTCACAATCAGCAGCATGGAGGCTGAAGATGCTGCCA
CTTATTACTGCCACCAGTATCATCGTTCCCCACCCACGTTCGGTGCTGGGACC
AAGCTGGAGCTGAAACGGGCTGATGCTGCAAAGCTT
Anti-human insulin scFV fusion with truncated asLOV2 at N-terminus (ntLOV)
(SEQ ID NO: 33; encoding SEQ ID NO: 18)
CATATGGGCGAATTTCTGGCGACCACCCTGGAACGCATTGAAAAAAACTTTG
TGATTACCGATCCGCGCCTGCCGGATAACCCGATTATTTTTGCGAGCGATAGC
TTTCTGCAGCTGACCGAATATAGCCGCGAAGAAATTCTGGGCCGCAACTGCC
GCTTTCTGCAGGGCCCGGAAACCGATCGCGCGACCGTGCGCAAAATTCGCGA
TGCGATTGATAACCAGACCGAAGTGACCGTGCAGCTGATTAACTATACCAAA
AGCGGCAAAAAATTTTGGAACCTGTTTCATCTGCAGCCGATGCGCGATCAGA
AAGGCGATGTGCAGTATTTTATTGGCGTGCAGCTGGATGGCACCGAACATGT
GCGCGATGCGGCGGAACGCGAAGCGGTGATGCTGATTAAAAAAACCGCGGC
ACAGATCCAGTTGGTGCAGTCTGGACCTGAACTGAAGAAGCCTGGAGAGACA
GTCAAGATCTCCTGCAAGGCTTCTGGTTATACCTTCACAGACTATTCAATGCA
CTGGGTGAAGCAGGCTCCAGGAAAGGGTTTAAAGTGGATGGACTGGATAAA
CACTGAGACTGGTGTGCCAACCTATGCAGATGACTTCAAGGGACGGTTTGCC
TTCTCTTTGGAAACCTCTGCTAACACTGCCTATTTGCAGATCAACGACCTCAA
AAATGAGGACACGGCTACATATTTCTGTACTAGAGGGTATGGAAAGGGGTAC
TTCGATGTCTGGGGCGCAGGGACCACGGTCACCGTCTCCTCAGCCAAAAGCA
CACCCCCATCCGTTTATCCATTGGCCCCTGGAAGCGGCGGAGGCGGATCAGG
AGGAGGAGGATCAGGCGGAGGAGGATCACAAATTGTTCTCACCCAGTCTCCA
ACAATCATGTCTGCATCTCTAGGGGAACGGGTCACCATGACCTGCACTGCCA
GCTCAAGTGTAAGTTCCAGTTACTTGCACTGGTACCAGCAGAAGCCAGGATC
CTCCCCCAAACTCTGGATTTATAGTACATCCAACCTGGCTTCTGGAGTCCCAG
CTCGCTTCAGTGGCAGTGGGTCTGGGACCTCTTACTCTCTCACAATCAGCAGC
ATGGAGGCTGAAGATGCTGCCACTTATTACTGCCACCAGTATCATCGTTCCCC
ACCCACGTTCGGTGCTGGGACCAAGCTGGAGCTGAAACGGGCTGATGCTGCA
AAGCTT
Anti-human insulin VH-asLOV2-VL with glycine-rich linkers (gsLOV)
(SEQ ID NO: 10)
ATGGCACAGATCCAGTTGGTGCAGTCTGGACCTGAACTGAAGAAGCCTGGAG
AGACAGTCAAGATCTCCTGCAAGGCTTCTGGTTATACCTTCACAGACTATTCA
ATGCACTGGGTGAAGCAGGCTCCAGGAAAGGGTTTAAAGTGGATGGACTGG
ATAAACACTGAGACTGGTGTGCCAACCTATGCAGATGACTTCAAGGGACGGT
TTGCCTTCTCTTTGGAAACCTCTGCTAACACTGCCTATTTGCAGATCAACGAC
CTCAAAAATGAGGACACGGCTACATATTTCTGTACTAGAGGGTATGGAAAGG
GGTACTTCGATGTCTGGGGCGCAGGGACCACGGTCACCGTCTCCTCAGCCAA
AAGCACACCCCCATCCGTTTATCCATTGGCCCCTGGAAGCGGCGGAGGCGGA
TCAGGCGAATTTCTGGCGACCACCCTGGAACGCATTGAAAAAAACTTTGTGA
TTACCGATCCGCGCCTGCCGGATAACCCGATTATTTTTGCGAGCGATAGCTTT
CTGCAGCTGACCGAATATAGCCGCGAAGAAATTCTGGGCCGCAACTGCCGCT
TTCTGCAGGGCCCGGAAACCGATCGCGCGACCGTGCGCAAAATTCGCGATGC
GATTGATAACCAGACCGAAGTGACCGTGCAGCTGATTAACTATACCAAAAGC
GGCAAAAAATTTTGGAACCTGTTTCATCTGCAGCCGATGCGCGATCAGAAAG
GCGATGTGCAGTATTTTATTGGCGTGCAGCTGGATGGCACCGAACATGTGCG
CGATGCGGCGGAACGCGAAGCGGTGATGCTGATTAAAAAAACCGCGGAAGA
AATTGATGAAGCGGCGGGCGGAGGAGGATCACAAATTGTTCTCACCCAGTCT
CCAACAATCATGTCTGCATCTCTAGGGGAACGGGTCACCATGACCTGCACTG
CCAGCTCAAGTGTAAGTTCCAGTTACTTGCACTGGTACCAGCAGAAGCCAGG
ATCCTCCCCCAAACTCTGGATTTATAGTACATCCAACCTGGCTTCTGGAGTCC
CAGCTCGCTTCAGTGGCAGTGGGTCTGGGACCTCTTACTCTCTCACAATCAGC
AGCATGGAGGCTGAAGATGCTGCCACTTATTACTGCCACCAGTATCATCGTTC
CCCACCCACGTTCGGTGCTGGGACCAAGCTGGAGCTGAAACGGGCTGATGCT
GCA;
or
(SEQ ID NO: 34; encoding SEQ ID NO: 19)
CATATGGCACAGATCCAGTTGGTGCAGTCTGGACCTGAACTGAAGAAGCCTG
GAGAGACAGTCAAGATCTCCTGCAAGGCTTCTGGTTATACCTTCACAGACTAT
TCAATGCACTGGGTGAAGCAGGCTCCAGGAAAGGGTTTAAAGTGGATGGACT
GGATAAACACTGAGACTGGTGTGCCAACCTATGCAGATGACTTCAAGGGACG
GTTTGCCTTCTCTTTGGAAACCTCTGCTAACACTGCCTATTTGCAGATCAACG
ACCTCAAAAATGAGGACACGGCTACATATTTCTGTACTAGAGGGTATGGAAA
GGGGTACTTCGATGTCTGGGGCGCAGGGACCACGGTCACCGTCTCCTCAGCC
AAAAGCACACCCCCATCCGTTTATCCATTGGCCCCTGGAAGCGGCGGAGGCG
GATCAGGCGAATTTCTGGCGACCACCCTGGAACGCATTGAAAAAAACTTTGT
GATTACCGATCCGCGCCTGCCGGATAACCCGATTATTTTTGCGAGCGATAGCT
TTCTGCAGCTGACCGAATATAGCCGCGAAGAAATTCTGGGCCGCAACTGCCG
CTTTCTGCAGGGCCCGGAAACCGATCGCGCGACCGTGCGCAAAATTCGCGAT
GCGATTGATAACCAGACCGAAGTGACCGTGCAGCTGATTAACTATACCAAAA
GCGGCAAAAAATTTTGGAACCTGTTTCATCTGCAGCCGATGCGCGATCAGAA
AGGCGATGTGCAGTATTTTATTGGCGTGCAGCTGGATGGCACCGAACATGTG
CGCGATGCGGCGGAACGCGAAGCGGTGATGCTGATTAAAAAAACCGCGGAA
GAAATTGATGAAGCGGCGAAAGAACTGGGCGGAGGCGGATCACAAATTGTT
CTCACCCAGTCTCCAACAATCATGTCTGCATCTCTAGGGGAACGGGTCACCAT
GACCTGCACTGCCAGCTCAAGTGTAAGTTCCAGTTACTTGCACTGGTACCAGC
AGAAGCCAGGATCCTCCCCCAAACTCTGGATTTATAGTACATCCAACCTGGC
TTCTGGAGTCCCAGCTCGCTTCAGTGGCAGTGGGTCTGGGACCTCTTACTCTC
TCACAATCAGCAGCATGGAGGCTGAAGATGCTGCCACTTATTACTGCCACCA
GTATCATCGTTCCCCACCCACGTTCGGTGCTGGGACCAAGCTGGAGCTGAAA
CGGGCTGATGCTGCAAAGCTT
Anti-human insulin V_{H}-asLOV2-V_{L} without glycine-rich linkers (nolinkLOV)
(SEQ ID NO: 35; encoding SEQ ID NO: 20)
CATATGGCACAGATCCAGTTGGTGCAGTCTGGACCTGAACTGAAGAAGCCTG
GAGAGACAGTCAAGATCTCCTGCAAGGCTTCTGGTTATACCTTCACAGACTAT
TCAATGCACTGGGTGAAGCAGGCTCCAGGAAAGGGTTTAAAGTGGATGGACT
GGATAAACACTGAGACTGGTGTGCCAACCTATGCAGATGACTTCAAGGGACG
GTTTGCCTTCTCTTTGGAAACCTCTGCTAACACTGCCTATTTGCAGATCAACG
ACCTCAAAAATGAGGACACGGCTACATATTTCTGTACTAGAGGGTATGGAAA
GGGGTACTTCGATGTCTGGGGCGCAGGGACCACGGTCACCGTCTCCTCAGCC
AAAAGCACACCCCCATCCGTTTATCCATTGGCCCCTGGAAGCGGCGAATTTCT
GGCGACCACCCTGGAACGCATTGAAAAAAACTTTGTGATTACCGATCCGCGC
CTGCCGGATAACCCGATTATTTTTGCGAGCGATAGCTTTCTGCAGCTGACCGA
ATATAGCCGCGAAGAAATTCTGGGCCGCAACTGCCGCTTTCTGCAGGGCCCG
GAAACCGATCGCGCGACCGTGCGCAAAATTCGCGATGCGATTGATAACCAGA
CCGAAGTGACCGTGCAGCTGATTAACTATACCAAAAGCGGCAAAAAATTTTG
GAACCTGTTTCATCTGCAGCCGATGCGCGATCAGAAAGGCGATGTGCAGTAT
TTTATTGGCGTGCAGCTGGATGGCACCGAACATGTGCGCGATGCGGCGGAAC
GCGAAGCGGTGATGCTGATTAAAAAAACCGCGGAAGAAATTGATGAAGCGG
CGAAAGAACTGCAAATTGTTCTCACCCAGTCTCCAACAATCATGTCTGCATCT
CTAGGGGAACGGGTCACCATGACCTGCACTGCCAGCTCAAGTGTAAGTTCCA
GTTACTTGCACTGGTACCAGCAGAAGCCAGGATCCTCCCCCAAACTCTGGAT
TTATAGTACATCCAACCTGGCTTCTGGAGTCCCAGCTCGCTTCAGTGGCAGTG
GGTCTGGGACCTCTTACTCTCTCACAATCAGCAGCATGGAGGCTGAAGATGC
TGCCACTTATTACTGCCACCAGTATCATCGTTCCCCACCCACGTTCGGTGCTG
GGACCAAGCTGGAGCTGAAACGGGCTGATGCTGCAAAGCTT

The nucleotide sequence encoding the anti-insulin scFv disclosed herein is provided in SEQ ID NO: 12. The nucleotide sequence encoding the V_{H} region of the anti-insulin scFV disclosed herein is provided in SEQ ID NO: 13. The nucleotide sequence encoding the V_{L} region of the anti-insulin scFV disclosed herein is provided in SEQ ID NO: 14.

Anti-Insulin scFv (Clone HB125)
(SEQ ID NO: 12)
ATGGCACAGATCCAGTTGGTGCAGTCTGGACCTGAACTGAAGAAGCCTG
GAGAGACAGTCAAGATCTCCTGCAAGGCTTCTGGTTATACCTTCACAGA
CTATTCAATGCACTGGGTGAAGCAGGCTCCAGGAAAGGGTTTAAAGTGG
ATGGACTGGATAAACACTGAGACTGGTGTGCCAACCTATGCAGATGACT
TCAAGGGACGGTTTGCCTTCTCTTTGGAAACCTCTGCTAACACTGCCTA
TTTGCAGATCAACGACCTCAAAAATGAGGACACGGCTACATATTTCTGT
ACTAGAGGGTATGGAAAGGGGTACTTCGATGTCTGGGGCGCAGGGACCA
CGGTCACCGTCTCCTCAGCCAAAAGCACACCCCCATCCGTTTATCCATT
GGCCCCTGGAAGCGGCGGAGGCGGATCAGGAGGAGGAGGATCAGGCGGA
GGAGGATCACAAATTGTTCTCACCCAGTCTCCAACAATCATGTCTGCAT
CTCTAGGGGAACGGGTCACCATGACCTGCACTGCCAGCTCAAGTGTAAG
TTCCAGTTACTTGCACTGGTACCAGCAGAAGCCAGGATCCTCCCCCAAA
CTCTGGATTTATAGTACATCCAACCTGGCTTCTGGAGTCCCAGCTCGCT
TCAGTGGCAGTGGGTCTGGGACCTCTTACTCTCTCACAATCAGCAGCAT
GGAGGCTGAAGATGCTGCCACTTATTACTGCCACCAGTATCATCGTTCC
CCACCCACGTTCGGTGCTGGGACCAAGCTGGAGCTGAAACGGGCTGATG
CTGCA
V_{H} region of anti-human insulin (Clone HB125)
(SEQ ID NO: 13)
ATGGCACAGATCCAGTTGGTGCAGTCTGGACCTGAACTGAAGAAGCCTG
GAGAGACAGTCAAGATCTCCTGCAAGGCTTCTGGTTATACCTTCACAGA
CTATTCAATGCACTGGGTGAAGCAGGCTCCAGGAAAGGGTTTAAAGTGG
ATGGACTGGATAAACACTGAGACTGGTGTGCCAACCTATGCAGATGACT
TCAAGGGACGGTTTGCCTTCTCTTTGGAAACCTCTGCTAACACTGCCTA
TTTGCAGATCAACGACCTCAAAAATGAGGACACGGCTACATATTTCTGT
ACTAGAGGGTATGGAAAGGGGTACTTCGATGTCTGGGGCGCAGGGACCA
CGGTCACCGTCTCCTCAGCCAAAAGCACACCCCCATCCGTTTATCCATT
GGCCCCTGGAAGC
V_{L} region of anti-human insulin (Clone HB125)
(SEQ ID NO: 14)
CAAATTGTTCTCACCCAGTCTCCAACAATCATGTCTGCATCTCTAGGGG
AACGGGTCACCATGACCTGCACTGCCAGCTCAAGTGTAAGTTCCAGTTA
CTTGCACTGGTACCAGCAGAAGCCAGGATCCTCCCCCAAACTCTGGATT
TATAGTACATCCAACCTGGCTTCTGGAGTCCCAGCTCGCTTCAGTGGCA
GTGGGTCTGGGACCTCTTACTCTCTCACAATCAGCAGCATGGAGGCTGA
AGATGCTGCCACTTATTACTGCCACCAGTATCATCGTTCCCCACCCACG
TTCGGTGCTGGGACCAAGCTGGAGCTGAAACGGGCTGATGCTGCA

The nucleotide sequence encoding asLOV2 is provided in SEQ ID NO: 15.

LOV2 derived from Avena sativa (asLOV2)
(SEQ ID NO: 15)
GGCGAATTTCTGGCGACCACCCTGGAACGCATTGAAAAAAACTTTGTGA
TTACCGATCCGCGCCTGCCGGATAACCCGATTATTTTTGCGAGCGATAG
CTTTCTGCAGCTGACCGAATATAGCCGCGAAGAAATTCTGGGCCGCAAC
TGCCGCTTTCTGCAGGGCCCGGAAACCGATCGCGCGACCGTGCGCAAAA
TTCGCGATGCGATTGATAACCAGACCGAAGTGACCGTGCAGCTGATTAA
CTATACCAAAAGCGGCAAAAAATTTTGGAACCTGTTTCATCTGCAGCCG
ATGCGCGATCAGAAAGGCGATGTGCAGTATTTTATTGGCGTGCAGCTGG
ATGGCACCGAACATGTGCGCGATGCGGCGGAACGCGAAGCGGTGATGCT
GATTAAAAAAACCGCGGAAGAAATTGATGAAGCGGCGAAAGAACTG

### Vectors and Host Cells

In another embodiment, provided herein is a vector comprising the engineered antibody fusion protein-encoding polynucleotide or a host cell expressing the vector comprising the engineered antibody fusion protein-encoding polynucleotide. Engineered antibody fusion proteins may be prepared by inserting an engineered or mutant polynucleotide into an appropriate expression vector and introducing the vector into an appropriate host cell, such as, for example, Escherichia coli. The transformant is cultured and the engineered antibody fusion protein expressed in the transformant may be collected from the cells or culture medium by any known technique.

In some embodiments the expression vector is a pET-30c vector (SEQ ID NO: 11).

pET-30c Expression Vector
(SEQ ID NO: 11)
ATCCGGATATAGTTCCTCCTTTCAGCAAAAAACCCCTCAAGACCCGTTT
AGAGGCCCCAAGGGGTTATGCTAGTTATTGCTCAGCGGTGGCAGCAGCC
AACTCAGCTTCCTTTCGGGCTTTGTTAGCAGCCGGATCTCAGTGGTGGT
GGTGGTGGTGCTCGAGTGCGGCCGCAAGCTTGTCGACGGAGCTCGAATT
CGGATCCACAGATATCCCATGGCCTTGTCGTCGTCGTCGGTACCCAGAT
CTGGGCTGTCCATGTGCTGGCGTTCGAATTTAGCAGCAGCGGTTTCTTT
CATACCAGAACCGCGTGGCACCAGACCAGAAGAATGATGATGATGATGG
TGCATATGTATATCTCCTTCTTAAAGTTAAACAAAATTATTTCTAGAGG
GGAATTGTTATCCGCTCACAATTCCCCTATAGTGAGTCGTATTAATTTC
GCGGGATCGAGATCGATCTCGATCCTCTACGCCGGACGCATCGTGGCCG
GCATCACCGGCGCCACAGGTGCGGTTGCTGGCGCCTATATCGCCGACAT
CACCGATGGGGAAGATCGGGCTCGCCACTTCGGGCTCATGAGCGCTTGT
TTCGGCGTGGGTATGGTGGCAGGCCCCGTGGCCGGGGGACTGTTGGGCG
CCATCTCCTTGCATGCACCATTCCTTGCGGCGGCGGTGCTCAACGGCCT
CAACCTACTACTGGGCTGCTTCCTAATGCAGGAGTCGCATAAGGGAGAG
CGTCGAGATCCCGGACACCATCGAATGGCGCAAAACCTTTCGCGGTATG
GCATGATAGCGCCCGGAAGAGAGTCAATTCAGGGTGGTGAATGTGAAAC
CAGTAACGTTATACGATGTCGCAGAGTATGCCGGTGTCTCTTATCAGAC
CGTTTCCCGCGTGGTGAACCAGGCCAGCCACGTTTCTGCGAAAACGCGG
GAAAAAGTGGAAGCGGCGATGGCGGAGCTGAATTACATTCCCAACCGCG
TGGCACAACAACTGGCGGGCAAACAGTCGTTGCTGATTGGCGTTGCCAC
CTCCAGTCTGGCCCTGCACGCGCCGTCGCAAATTGTCGCGGCGATTAAA
TCTCGCGCCGATCAACTGGGTGCCAGCGTGGTGGTGTCGATGGTAGAAC
GAAGCGGCGTCGAAGCCTGTAAAGCGGCGGTGCACAATCTTCTCGCGCA
ACGCGTCAGTGGGCTGATCATTAACTATCCGCTGGATGACCAGGATGCC
ATTGCTGTGGAAGCTGCCTGCACTAATGTTCCGGCGTTATTTCTTGATG
TCTCTGACCAGACACCCATCAACAGTATTATTTTCTCCCATGAAGACGG
TACGCGACTGGGCGTGGAGCATCTGGTCGCATTGGGTCACCAGCAAATC
GCGCTGTTAGCGGGCCCATTAAGTTCTGTCTCGGCGCGTCTGCGTCTGG
CTGGCTGGCATAAATATCTCACTCGCAATCAAATTCAGCCGATAGCGGA
ACGGGAAGGCGACTGGAGTGCCATGTCCGGTTTTCAACAAACCATGCAA
ATGCTGAATGAGGGCATCGTTCCCACTGCGATGCTGGTTGCCAACGATC
AGATGGCGCTGGGCGCAATGCGCGCCATTACCGAGTCCGGGCTGCGCGT
TGGTGCGGACATCTCGGTAGTGGGATACGACGATACCGAAGACAGCTCA
TGTTATATCCCGCCGTTAACCACCATCAAACAGGATTTTCGCCTGCTGG
GGCAAACCAGCGTGGACCGCTTGCTGCAACTCTCTCAGGGCCAGGCGGT
GAAGGGCAATCAGCTGTTGCCCGTCTCACTGGTGAAAAGAAAAACCACC
CTGGCGCCCAATACGCAAACCGCCTCTCCCCGCGCGTTGGCCGATTCAT
TAATGCAGCTGGCACGACAGGTTTCCCGACTGGAAAGCGGGCAGTGAGC
GCAACGCAATTAATGTAAGTTAGCTCACTCATTAGGCACCGGGATCTCG
ACCGATGCCCTTGAGAGCCTTCAACCCAGTCAGCTCCTTCCGGTGGGCG
CGGGGCATGACTATCGTCGCCGCACTTATGACTGTCTTCTTTATCATGC
AACTCGTAGGACAGGTGCCGGCAGCGCTCTGGGTCATTTTCGGCGAGGA
CCGCTTTCGCTGGAGCGCGACGATGATCGGCCTGTCGCTTGCGGTATTC
GGAATCTTGCACGCCCTCGCTCAAGCCTTCGTCACTGGTCCCGCCACCA
AACGTTTCGGCGAGAAGCAGGCCATTATCGCCGGCATGGCGGCCCCACG
GGTGCGCATGATCGTGCTCCTGTCGTTGAGGACCCGGCTAGGCTGGCGG
GGTTGCCTTACTGGTTAGCAGAATGAATCACCGATACGCGAGCGAACGT
GAAGCGACTGCTGCTGCAAAACGTCTGCGACCTGAGCAACAACATGAAT
GGTCTTCGGTTTCCGTGTTTCGTAAAGTCTGGAAACGCGGAAGTCAGCG
CCCTGCACCATTATGTTCCGGATCTGCATCGCAGGATGCTGCTGGCTAC
CCTGTGGAACACCTACATCTGTATTAACGAAGCGCTGGCATTGACCCTG
AGTGATTTTTCTCTGGTCCCGCCGCATCCATACCGCCAGTTGTTTACCC
TCACAACGTTCCAGTAACCGGGCATGTTCATCATCAGTAACCCGTATCG
TGAGCATCCTCTCTCGTTTCATCGGTATCATTACCCCCATGAACAGAAA
TCCCCCTTACACGGAGGCATCAGTGACCAAACAGGAAAAAACCGCCCTT
AACATGGCCCGCTTTATCAGAAGCCAGACATTAACGCTTCTGGAGAAAC
TCAACGAGCTGGACGCGGATGAACAGGCAGACATCTGTGAATCGCTTCA
CGACCACGCTGATGAGCTTTACCGCAGCTGCCTCGCGCGTTTCGGTGAT
GACGGTGAAAACCTCTGACACATGCAGCTCCCGGAGACGGTCACAGCTT
GTCTGTAAGCGGATGCCGGGAGCAGACAAGCCCGTCAGGGCGCGTCAGC
GGGTGTTGGCGGGTGTCGGGGCGCAGCCATGACCCAGTCACGTAGCGAT
AGCGGAGTGTATACTGGCTTAACTATGCGGCATCAGAGCAGATTGTACT
GAGAGTGCACCATATATGCGGTGTGAAATACCGCACAGATGCGTAAGGA
GAAAATACCGCATCAGGCGCTCTTCCGCTTCCTCGCTCACTGACTCGCT
GCGCTCGGTCGTTCGGCTGCGGCGAGCGGTATCAGCTCACTCAAAGGCG
GTAATACGGTTATCCACAGAATCAGGGGATAACGCAGGAAAGAACATGT
GAGCAAAAGGCCAGCAAAAGGCCAGGAACCGTAAAAAGGCCGCGTTGCT
GGCGTTTTTCCATAGGCTCCGCCCCCCTGACGAGCATCACAAAAATCGA
CGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCT
CCAAGCTGGGCTGTGTGCACGAACCCCCCGTTCAGCCCGACCGCTGCGC
CTTATCCGGTAACTATCGTCTTGAGTCCAACCCGGTAAGACACGACTTA
TCGCCACTGGCAGCAGCCACTGGTAACAGGATTAGCAGAGCGAGGTATG
TAGGCGGTGCTACAGAGTTCTTGAAGTGGTGGCCTAACTACGGCTACAC
TAGAAGGACAGTATTTGGTATCTGCGCTCTGCTGAAGCCAGTTACCTTC
GGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGG
ATCTCAAGAAGATCCTTTGATCTTTTCTACGGGGTCTGACGCTCAGTGG
AACGAAAACTCACGTTAAGGGATTTTGGTCATGAACAATAAAACTGTCT
GCTTACATAAACAGTAATACAAGGGGTGTTATGAGCCATATTCAACGGG
AAACGTCTTGCTCTAGGCCGCGATTAAATTCCAACATGGATGCTGATTT
ATATGGGTATAAATGGGCTCGCGATAATGTCGGGCAATCAGGTGCGACA
ATCTATCGATTGTATGGGAAGCCCGATGCGCCAGAGTTGTTTCTGAAAC
ATGGCAAAGGTAGCGTTGCCAATGATGTTACAGATGAGATGGTCAGACT
AAACTGGCTGACGGAATTTATGCCTCTTCCGACCATCAAGCATTTTATC
CGTACTCCTGATGATGCATGGTTACTCACCACTGCGATCCCCGGGAAAA
CAGCATTCCAGGTATTAGAAGAATATCCTGATTCAGGTGAAAATATTGT
TGATGCGCTGGCAGTGTTCCTGCGCCGGTTGCATTCGATTCCTGTTTGT
AATTGTCCTTTTAACAGCGATCGCGTATTTCGTCTCGCTCAGGCGCAAT
CACGAATGAATAACGGTTTGGTTGATGCGAGTGATTTTGATGACGAGCG
TAATGGCTGGCCTGTTGAACAAGTCTGGAAAGAAATGCATAAACTTTTG
CCATTCTCACCGGATTCAGTCGTCACTCATGGTGATTTCTCACTTGATA
ACCTTATTTTTGACGAGGGGAAATTAATAGGTTGTATTGATGTTGGACG
AGTCGGAATCGCAGACCGATACCAGGATCTTGCCATCCTATGGAACTGC
CTCGGTGAGTTTTCTCCTTCATTACAGAAACGGCTTTTTCAAAAATATG
GTATTGATAATCCTGATATGAATAAATTGCAGTTTCATTTGATGCTCGA
TGAGTTTTTCTAAGAATTAATTCATGAGCGGATACATATTTGAATGTAT
TTAGAAAAATAAACAAATAGGGGTTCCGCGCACATTTCCCCGAAAAGTG
CCACCTGAAATTGTAAACGTTAATATTTTGTTAAAATTCGCGTTAAATT
TTTGTTAAATCAGCTCATTTTTTAACCAATAGGCCGAAATCGGCAAAAT
CCCTTATAAATCAAAAGAATAGACCGAGATAGGGTTGAGTGTTGTTCCA
GTTTGGAACAAGAGTCCACTATTAAAGAACGTGGACTCCAACGTCAAAG
GGCGAAAAACCGTCTATCAGGGCGATGGCCCACTACGTGAACCATCACC
CTAATCAAGTTTTTTGGGGTCGAGGTGCCGTAAAGCACTAAATCGGAAC
CCTAAAGGGAGCCCCCGATTTAGAGCTTGACGGGGAAAGCCGGCGAACG
TGGCGAGAAAGGAAGGGAAGAAAGCGAAAGGAGCGGGCGCTAGGGCGCT
GGCAAGTGTAGCGGTCACGCTGCGCGTAACCACCACACCCGCCGCGCTT
AATGCGCCGCTACAGGGCGCGTCCCATTCGCCA

In embodiments, the engineered antibody fusion protein thus obtained may be purified by any of the known purification techniques including, but not limited to, ion exchange column chromatography, affinity chromatography, liquid chromatography, filtration, ultrafiltration, salt precipitation, solvent precipitation, immunoprecipitation, gel electrophoresis, isoelectric electrophoresis and dialysis.

In embodiments, provided herein are isolated or purified polypeptides, proteins and polynucleotides for an engineered antibody fusion protein, a vector comprising the polynucleotide encoding the engineered antibody fusion protein, a host cell transformed with such a vector, and a method for preparing the engineered antibody fusion protein by culturing the transformant, collecting and purifying the engineered antibody fusion protein from the culture.

### III. Devices

In another embodiment, a device for assaying a target molecule in a sample is provided, where the device includes an engineered antibody fusion protein described herein.

Disclosed herein are biosensors comprising an engineered antibody fusion protein to detect a target molecule, wherein the binding affinity of the antibody fusion protein towards the target is modulated upon exposure to an external signal. The modulable binding affinity of the antibody fusion proteins disclosed herein allow for reversible binding to the target molecule, enabling continuous, in vivo monitoring applications.

As used herein, “biosensor” means a device containing bio-recognition elements (e.g., enzymes, aptamers, antibodies, etc.) that react with a target molecule and then transduce the biological signal into an electrical signal that can be analyzed.

In embodiments, an electrode comprises an antibody fusion protein disclosed herein immobilized on an electroconductive material. Examples of means for immobilizing molecules such as the engineered antibody fusion protein include, but are not limited to, cross-linking, encapsulating into a macromolecular matrix, coating with a dialysis membrane, optical cross-linking polymer, electroconductive polymer, oxidation-reduction polymer, and any combination thereof. The electroconductive material may be any material known in the art for biosensing applications.

The term “immobilized” refers to a compound which is attached to a surface (e.g., electrode). Immobilization can be performed through adsorption, cross-linking, covalent bonding and/or affinity-tag binding.

The term “cross-linking” refers to a polymerization reaction of a cross-linking reagent. A “cross-linking reagent” refers to a molecule that contains two or more reactive ends capable of forming covalent bonds.

The term “electroconductive material” refers to a substance capable of transmitting electricity.

In some embodiments, the biosensor comprising the antibody fusion protein disclosed herein includes a source of light to irradiate the antibody fusion protein to modulate the binding affinity of the antibody fusion protein for the target molecule. In some embodiments, the light source irradiates blue light, wherein the wavelength of the light may be about 400-700 nm.

In one embodiment, the biosensor detects a target molecule in a continuous manner. In one embodiment, the target molecule is insulin.

### REFERENCES

1. Kopka, B., et al. (2017). Electron transfer pathways in a light, oxygen, voltage (LOV) protein devoid of the photoactive cysteine. Scientific Reports, 7(1), 1-16. https://doi.org/10.1038/s41598-017-13420-1

### EXAMPLES

### Example 1: Characterization of an Antibody Fusion Protein with a Redox-Sensitive Domain

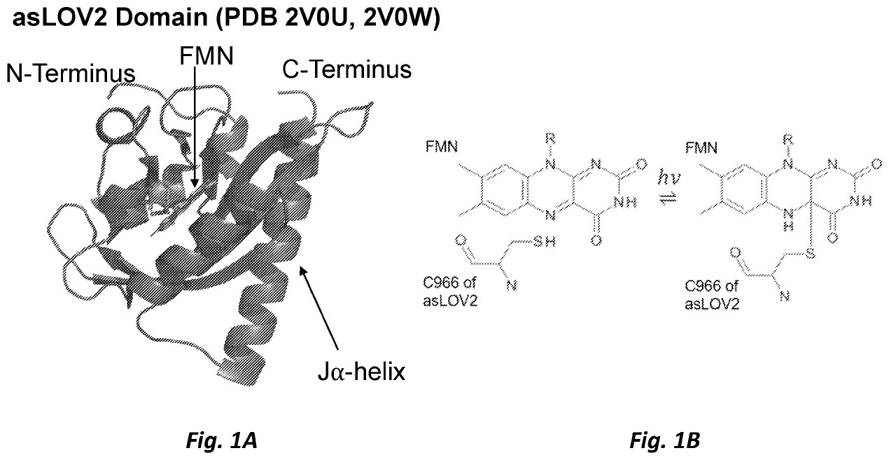

For improved biosensing properties, antibody fusion proteins were designed with a single chain variable fragment (scFv) of an immunoglobulin and a redox-sensitive domain capable of undergoing a conformational change upon exposure to light. The light oxygen voltage domain (LOV) derived from Avena sativa (asLOV2) was used as the initial redox-sensitive domain, as it has been extensively characterized and has been used for protein switches. The crystal structure of asLOV2 is shown in FIG. 1A, which includes a flavin mononucleotide (FMN) cofactor. As shown in FIG. 1B, upon irradiation with blue light the Cys966 residue of asLOV2 can form a covalent bond with the FMN, which results in the undocking of the Jα-helix of the C-terminus. Anti-insulin scFv (clone HB125) was chosen as a model target for this system, as insulin would be a desirable hormone to monitor with a biosensor for patients with diabetes.

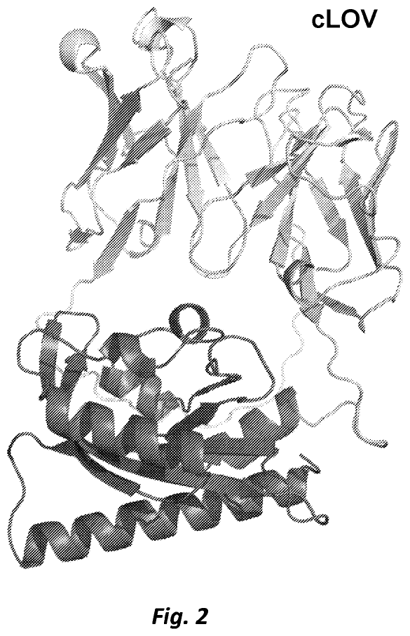

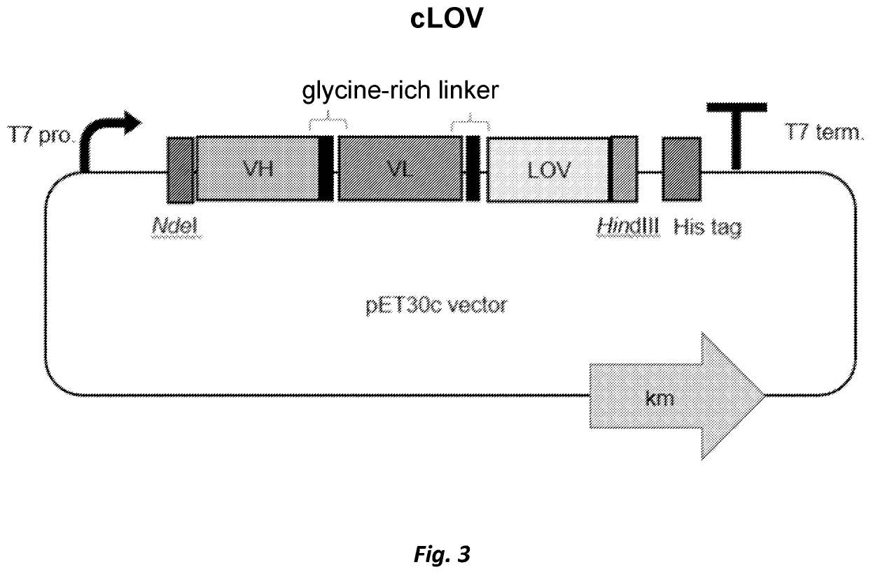

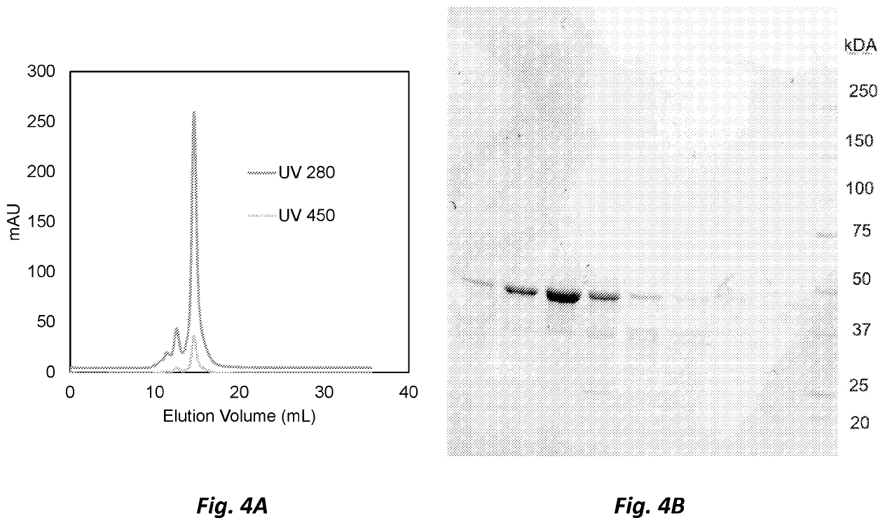

An antibody fusion protein was designed with an anti-human insulin scFv and asLOV2 domain at the C-terminus (cLOV). For the initial model, the antibody fusion protein was produced with the anti-human insulin scFv consisting of an N-terminal V_{H} region, followed by a glycine-rich linker, followed by the V_{L} region and the asLOV2 domain at the C-terminus (SEQ ID NO: 3). The structure of this antibody fusion protein as predicted by AlphaFold 2 is shown in FIG. 2, where the asLOV2 domain with its alpha helixes is seen at the bottom and the V_{H} and V_{L} regions are seen on the top of the structure. A pET30c vector with NdeI and HindIII restriction sites and a His tag was used to produce the recombinant antibody fusion protein in E. coli Shuffle T7 Express™ cells (FIG. 3). The antibody fusion protein was collected as a soluble fraction and its expression was confirmed by SDS-PAGE (FIG. 4B). The crude protein was purified by affinity chromatography using the His tag and by size exclusion chromatography (FIG. 4A).

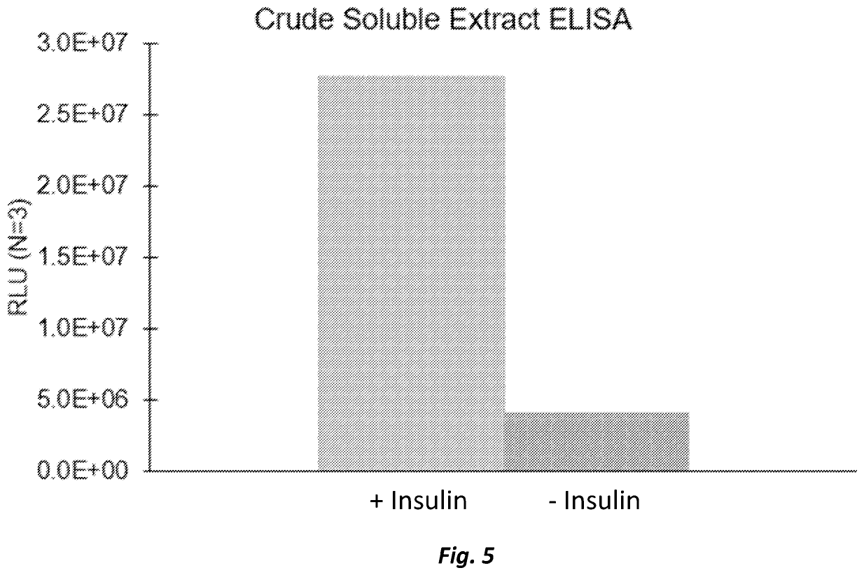

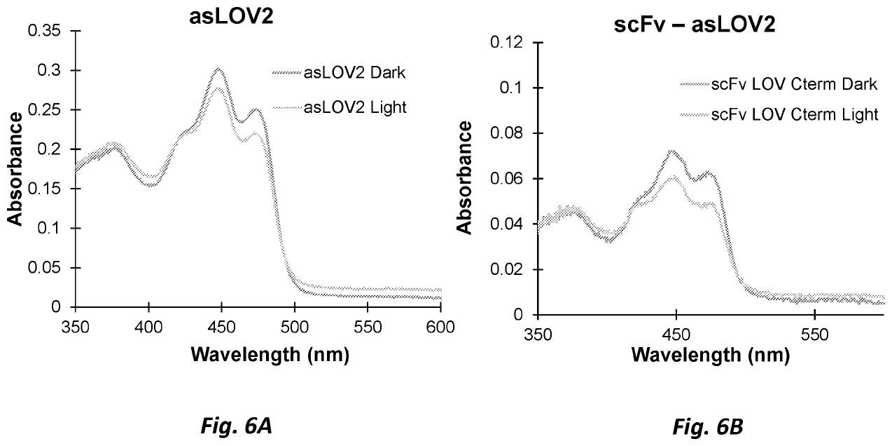

An insulin sandwich ELISA was performed using the antibody fusion protein (SEQ ID NO: 3) or the anti-insulin scFv (SEQ ID NO: 1) and an anti-His-tag IgG antibody conjugated to HRP for detection. The ELISA confirmed that the antibody fusion protein retained its ability to insulin (FIG. 5). Absorbance of the antibody fusion protein was measured in both dark and light conditions (FIG. 6A) and compared to the absorbance of an asLOV2 protein (FIG. 6B) without the scFV domain. For the dark condition, the absorbance was measured in a dark room. For the light condition, light exposure was performed using an LED array at 470 nm. This spectroscopic analysis revealed that the antibody fusion protein exhibited similar characteristic absorbance spectra to the asLOV2 protein, thus showing the antibody fusion protein retained its FMN activity.

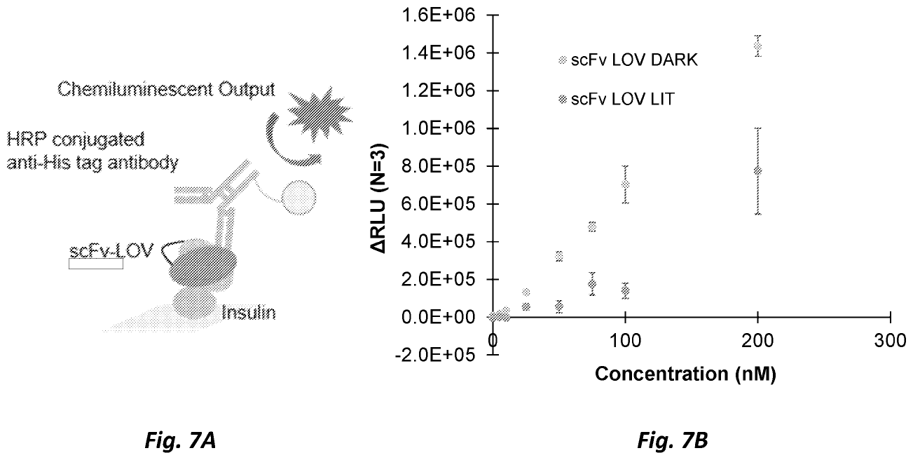

Then, another insulin sandwich ELISA (FIG. 7A) was performed with the antibody fusion protein in both light and dark conditions. For the ELISA assay, the entire procedure including all washing and incubation steps were performed either in a dark room, or under a LED array with constant 470 nm light exposure. Significantly different binding ability was observed in light vs. dark conditions at various concentrations of antibody, which indicates that the antibody fusion protein has a different binding affinity towards its substrate in the presence of light, which activates the LOV domain. FIG. 7B shows the sensitivity of the antibody fusion protein changed by approximately 2-fold in the presence of light, as observed by the slope of insulin detected by the concentration of the antibody. These results validate the hypothesis that an antibody fusion protein can have modulable binding towards its substrate based on the conformational change which occurs by activating a redox-sensitive domain with an external signal (e.g., blue light).

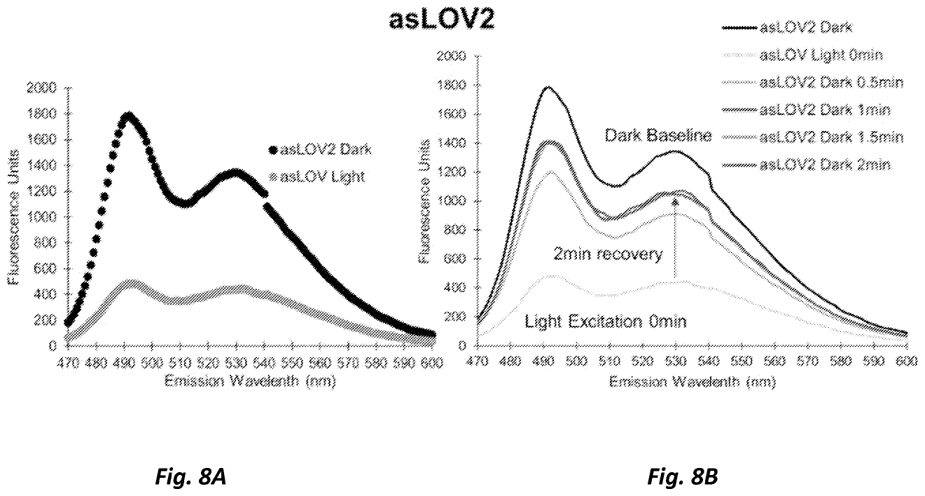

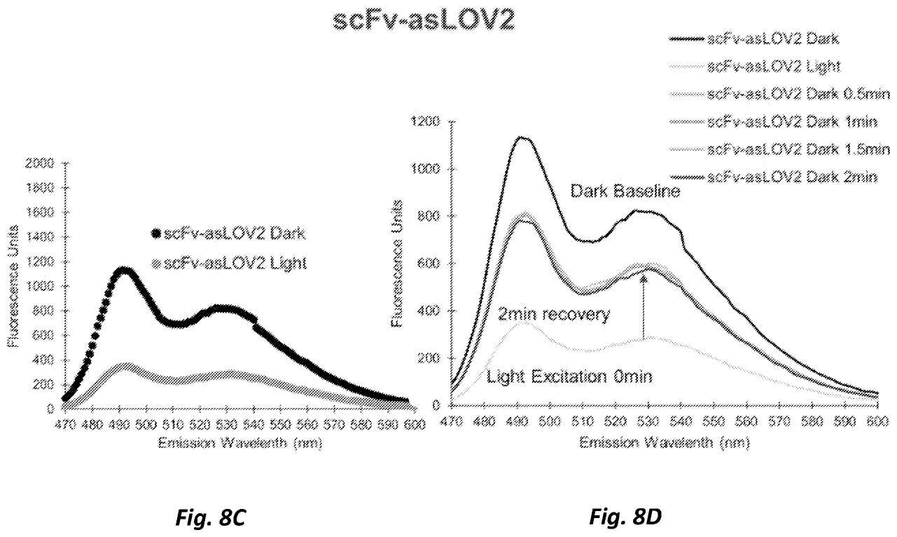

FIG. 8 shows further spectroscopic analysis of an asLOV protein and the antibody fusion with an anti-insulin scFv domain and an asLOV2 domain at the C-terminus (cLOV). Here, the intrinsic FMN fluorescence is measured. The antibody fusion protein (cLOV) exhibited a similar characteristic FMN spectra to the asLOV2 protein thus showing the antibody fusion protein retained its FMN activity.

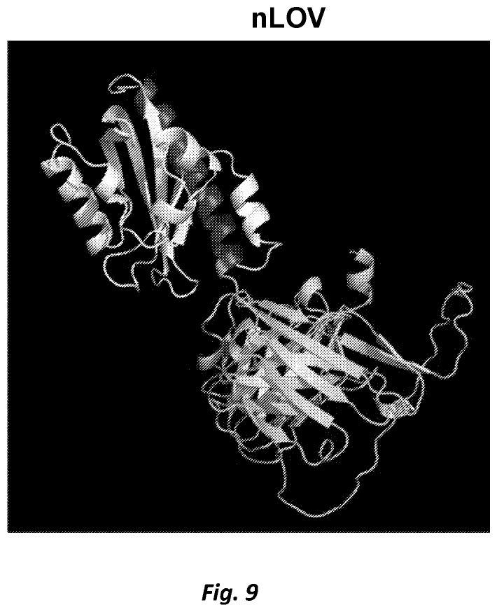

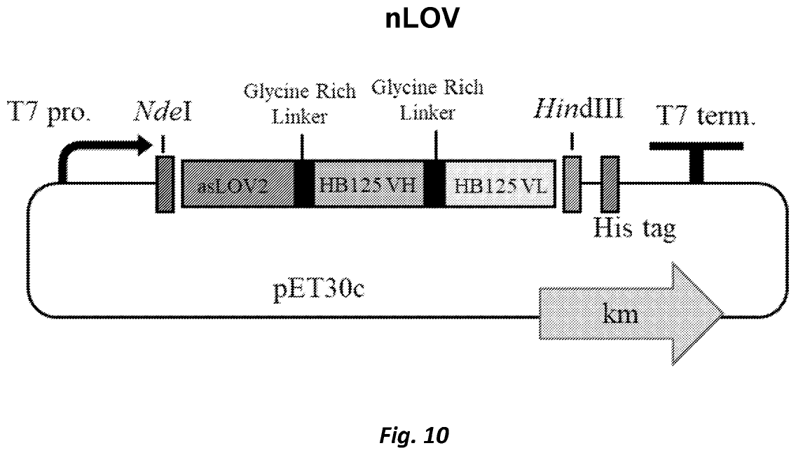

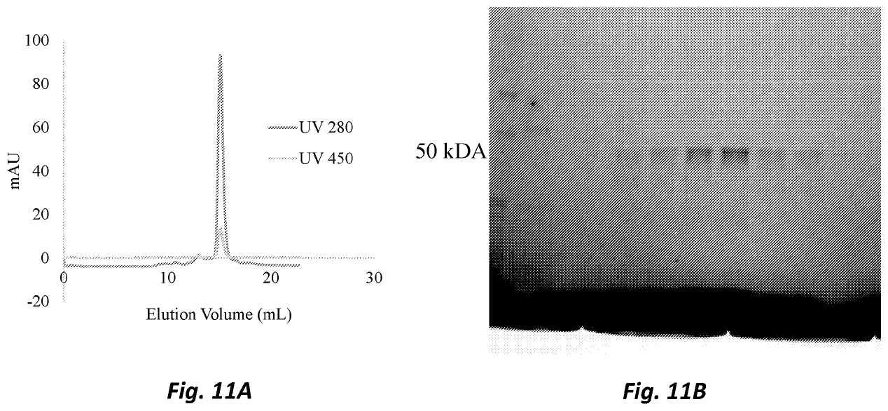

A second antibody fusion protein was designed with an anti-human insulin scFv and an asLOV2 domain at the N-terminus (nLOV). FIG. 9 shows the structure of the second engineered antibody fusion protein (nLOV) as predicted by AlphaFold 2. The alpha helix involved in the conformational change of the asLOV2 domain is shown in dark gray. FIG. 10 shows a pET30c vector with NdeI and HindIII restriction sites and a His tag was used to produce the second recombinant antibody fusion protein (nLOV) protein in E. coli Shuffle T7 Express™ cells (SEQ ID NO: 17). FIG. 11A shows the chromatogram tracking at 280 nm (UV280) and 450 nm (UV450) absorptions of the second antibody fusion (nLOV), and FIG. 11B shows SDS-PAGE of the eluted soluble antibody fusion protein with an anti-insulin scFV domain and an asLOV2 domain, which was expressed in E. coli Shuffle T7 Express™ cells.

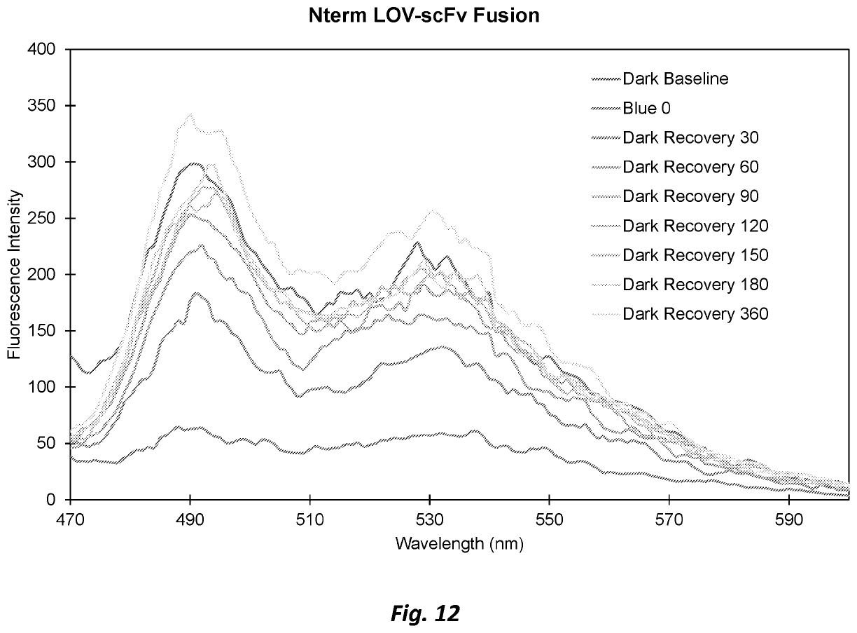

FIG. 12 shows the intrinsic FMN fluorescence of the antibody fusion protein (nLOV), measured in both dark and light conditions. For the dark condition, the absorbance was measured in a dark room. For the light condition, light exposure was performed using an LED array at 470 nm. The intrinsic FMN fluorescence signal was reversible over the course of three minutes, indicating the LOV protein retains its activity.

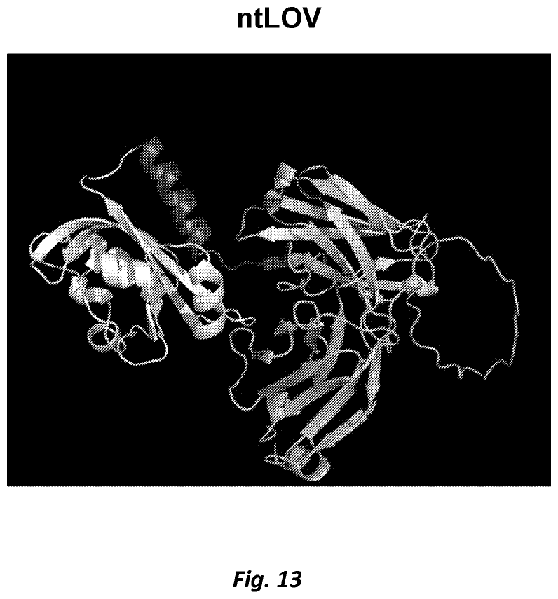

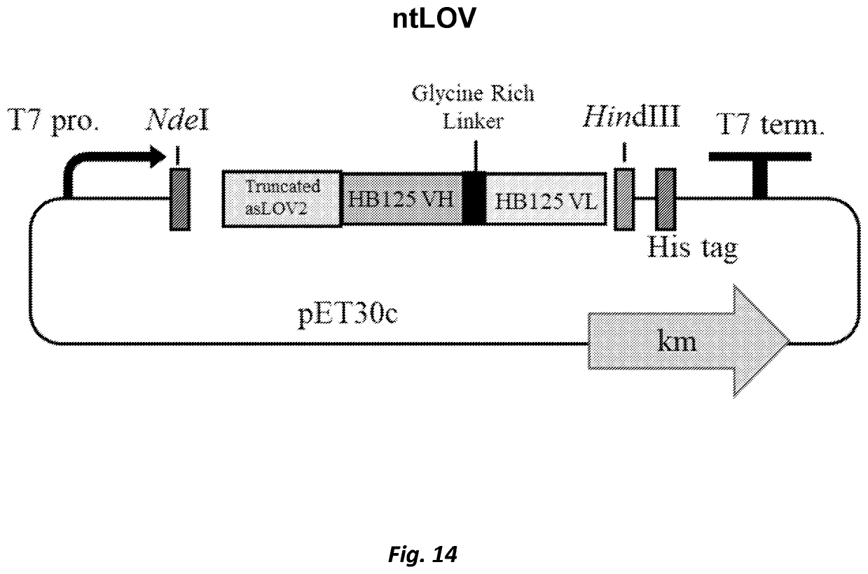

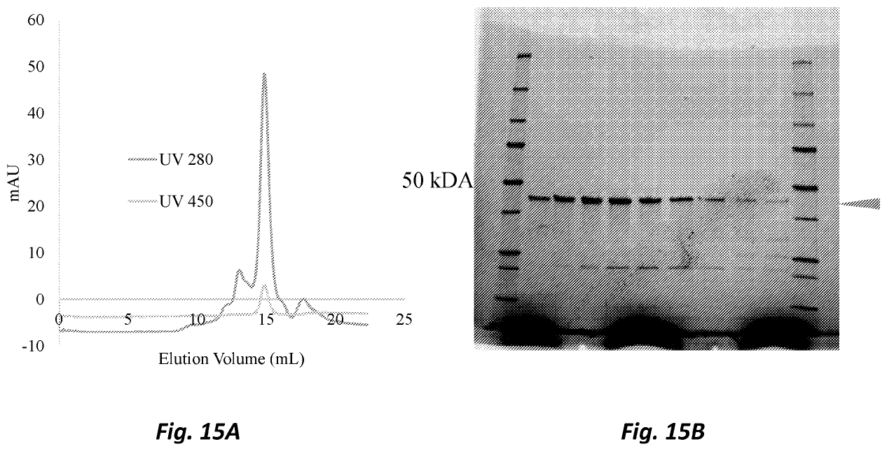

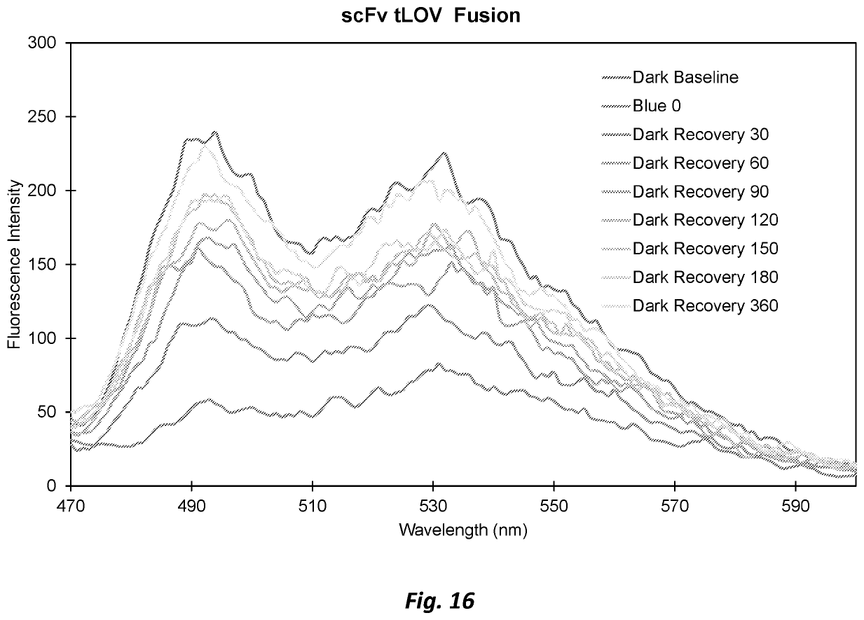

A third antibody fusion protein was designed with an anti-insulin single-chain variable fragment (scFV) domain and a truncated asLOV2 domain at the N-terminus. FIG. 13 shows the structure of the third engineered antibody fusion protein (ntLOV) as predicted by AlphaFold 2. The alpha helix involved in the conformational change of the asLOV2 domain is shown in dark gray. FIG. 14 shows a pET30c vector with NdeI and HindIII restriction sites and a His tag used to produce the third recombinant antibody fusion (ntLOV) protein in E. coli Shuffle T7 Express™ cells (SEQ ID NO: 18). FIG. 15A shows the chromatogram tracking at 280 nm (UV280) and 450 nm (UV450) absorptions of the third antibody fusion (ntLOV), and FIG. 15B shows SDS-PAGE of the eluted soluble third antibody fusion (ntLOV) expressed in E. coli Shuffle T7 Express™ cells. FIG. 16 shows the intrinsic FMN Fluorescence of the third antibody fusion protein (ntLOV), measured in both dark and light conditions. For the dark condition, the fluorescence was measured in a dark room. For the light condition, light exposure was performed using an LED array at 470 nm. The intrinsic FMN fluorescence signal was reversible over the course of three minutes, indicating the LOV protein retains its activity in this fusion.

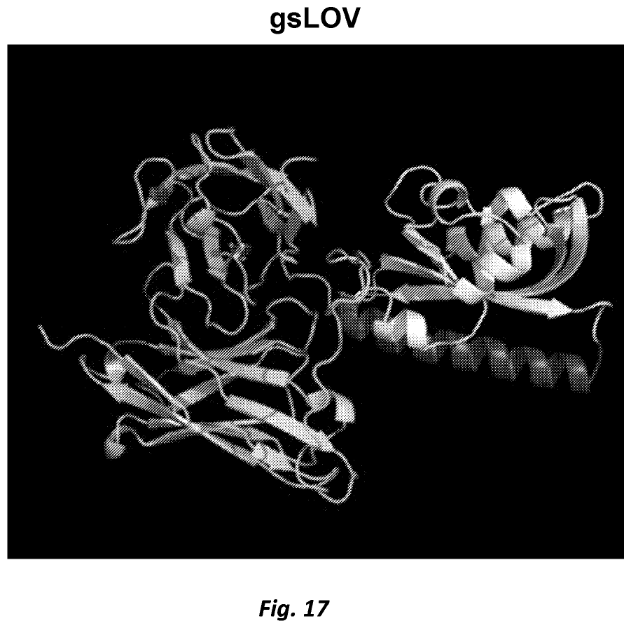

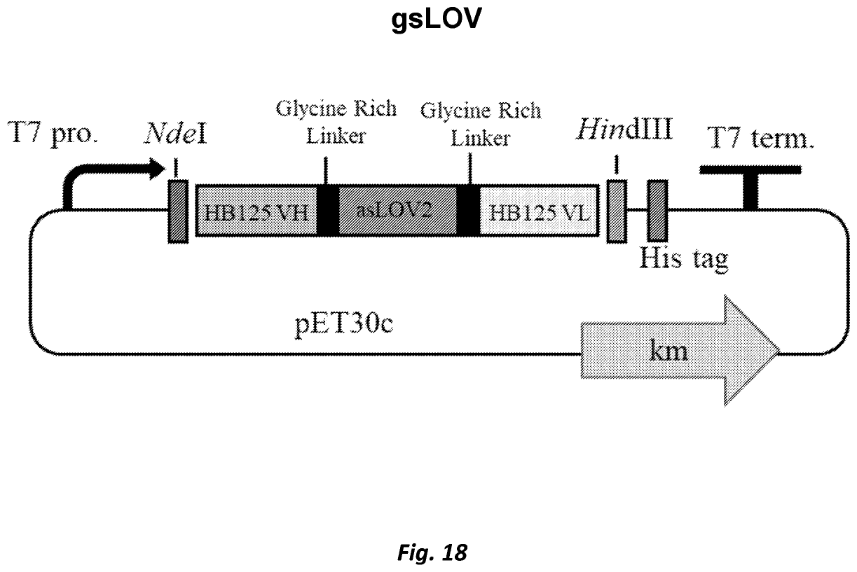

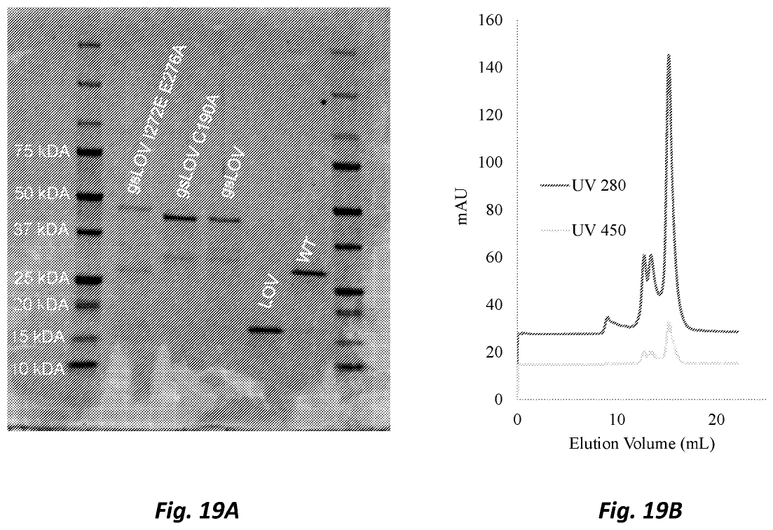

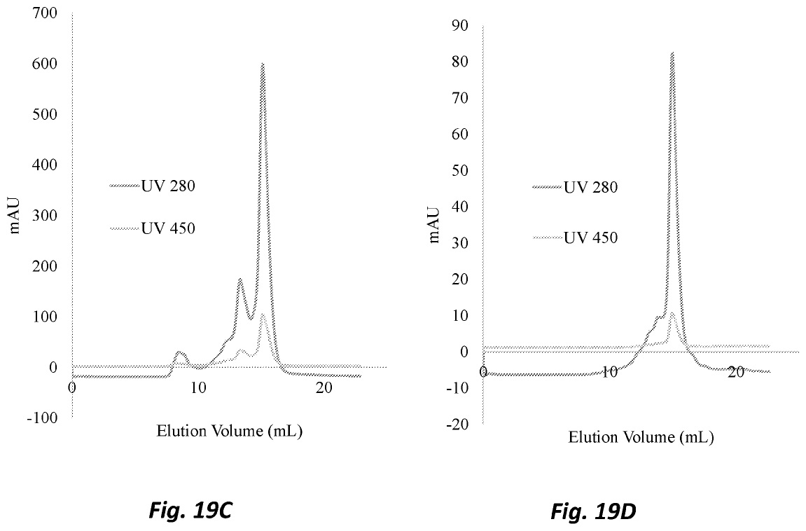

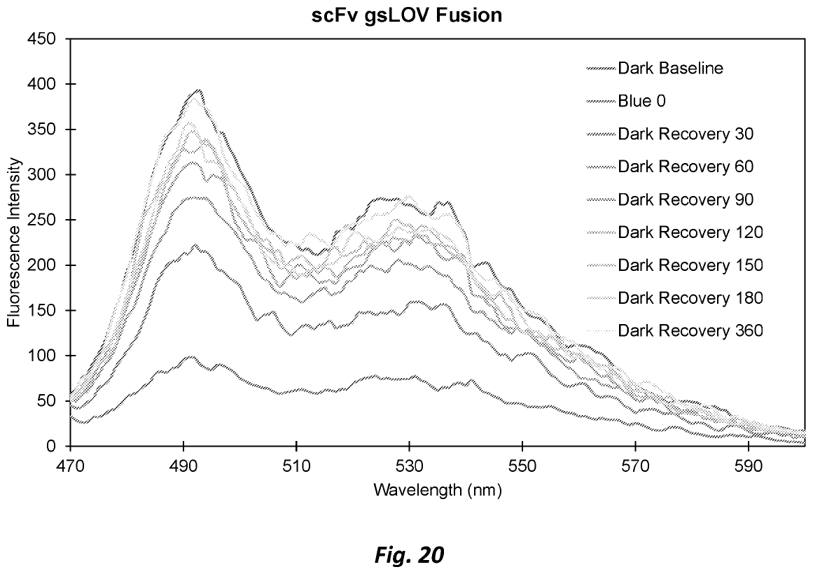

A fourth antibody fusion protein was designed with an asLOV2 domain between an anti-insulin V_{H} region at the N-terminus and an anti-insulin V_{L} region at the C-terminus with glycine-rich linkers between the domains (gsLOV). FIG. 17 shows the structure of the fourth engineered antibody fusion protein (gsLOV) as predicted by AlphaFold 2. The alpha helix involved in the conformational change of the asLOV2 domain is shown in dark gray. FIG. 18 shows a pET30c vector with NdeI and HindIII restriction sites and a His tag was used to produce the fourth recombinant antibody fusion protein (gsLOV) in E. coli Shuffle T7 Express™ cells (SEQ ID NO: 19). Variants of the gsLOV fusion proteins were prepared with mutations to fix the proteins in a “dark state” (C190A; SEQ ID NO: 27) or a “light state” (1272E, A276E; SEQ ID NO: 26). FIG. 19A shows SDS-PAGE of the eluted soluble gsLOV antibody fusion proteins expressed in E. coli Shuffle T7 Express™ cells. FIGS. 19B-19D show the chromatogram tracking at 280 nm (UV280) and 450 nm (UV450) absorptions for the gsLOV (FIG. 19B), the gsLOV C190A “dark” variant (FIG. 19C), and the gsLOV I272E, A276E “light” variant (FIG. 19D) antibody fusion proteins. FIG. 20 shows the intrinsic FMN Fluorescence of the gsLOV antibody fusion protein, measured in both dark and light conditions. For the dark condition, the absorbance was measured in a dark room. For the light condition, light exposure was performed using an LED array at 470 nm. The intrinsic FMN fluorescence signal was reversible over the course of three minutes, indicating the LOV protein retains its activity in this fusion.

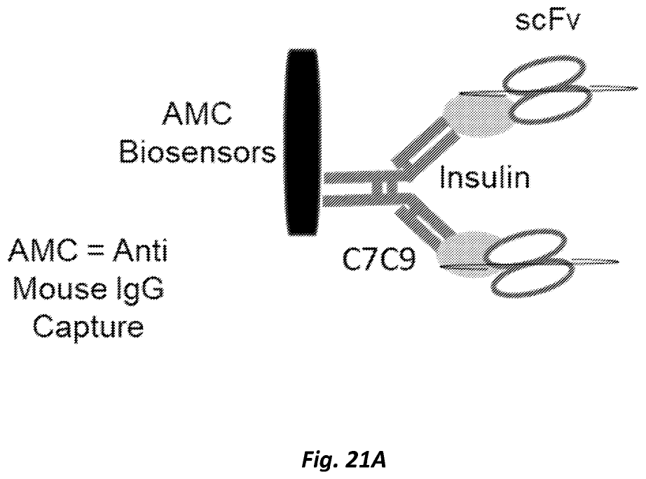

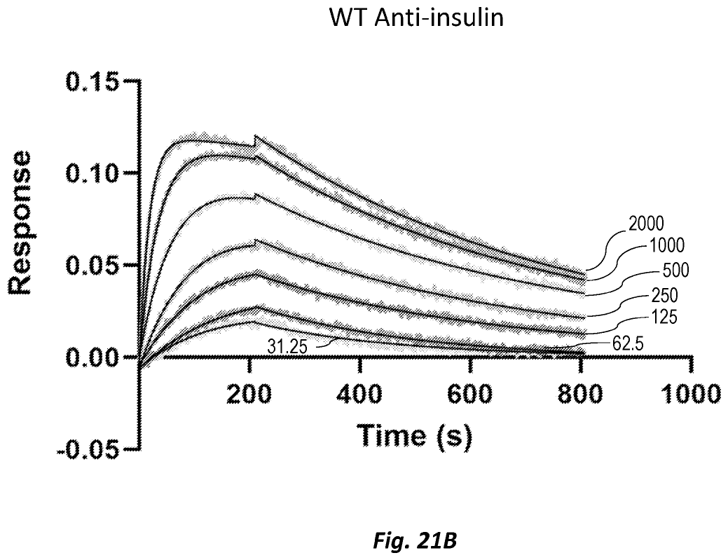

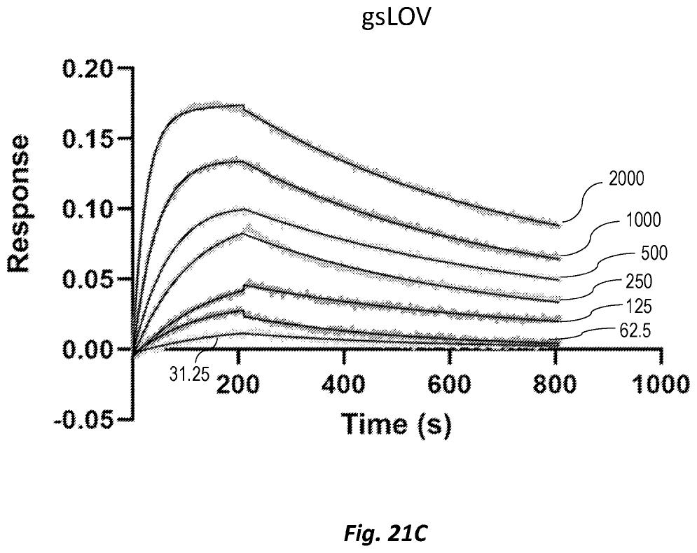

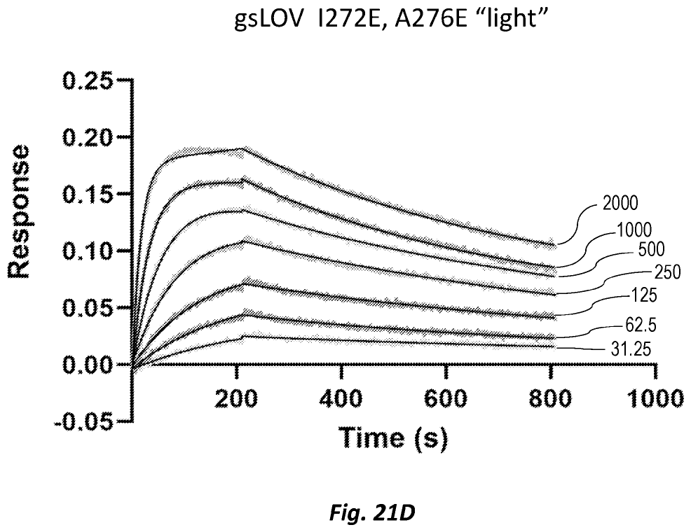

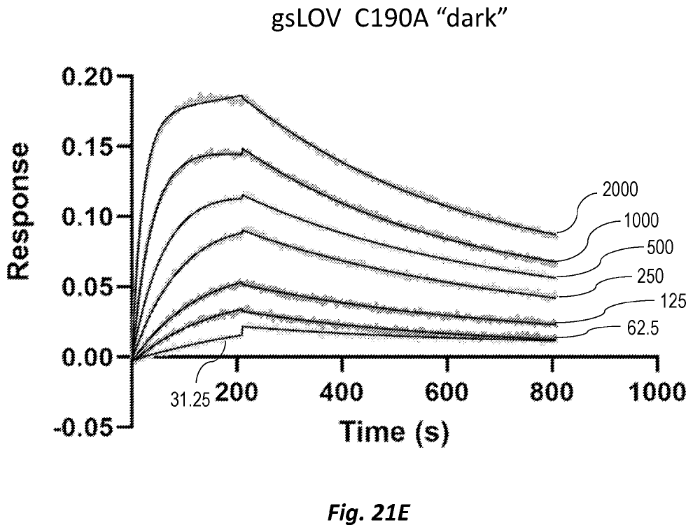

A Biolayer Interferometry (BLI) assay was performed to determine the binding affinity of the gsLOV antibody fusion proteins to insulin, as shown in FIG. 21A. FIGS. 21B-21E show representative sensor grams from the BLI assay for the wild type anti-insulin (FIG. 21B), the gsLOV fusion protein (FIG. 21C), the gsLOV I272E, A276E fusion protein fixed in a “light state” (FIG. 21D), and the gsLOV C190A fusion protein fixed in a “dark state” (FIG. 21E). The corresponding calculated affinities, shown in Table 1, reveal that the binding affinity of the dark mimetic fusion is twice that of the light mimetic fusion, which is comparable to the wild-type anti-insulin.

TABLE 1
Binding affinities of antibody fusion proteins with an
asLOV2 domain between an anti-insulin V_{II} region at the
N-terminus and an anti-insulin V_{L} region at the C-terminus
with glycine-rich linkers (gsLOV)

| | K_{D} | Kₒₙ | K_{off} |
|---|---|---|---|
| Sample | (nM) | (M⁻¹ * min⁻¹) | (min⁻¹) |
| WT anti-insulin (HB125) | 55.2 | 26790 | 0.001479 |
| gsLOV | 100.4 | 19645 | 0.001972 |
| gsLOV C190A “dark” | 103.7 | 23397 | 0.002426 |
| gsLOV I272E, A276E “light” | 63.84 | 29514 | 0.001885 |

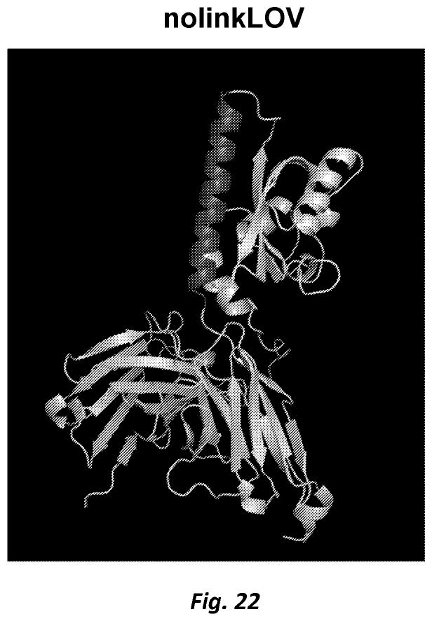

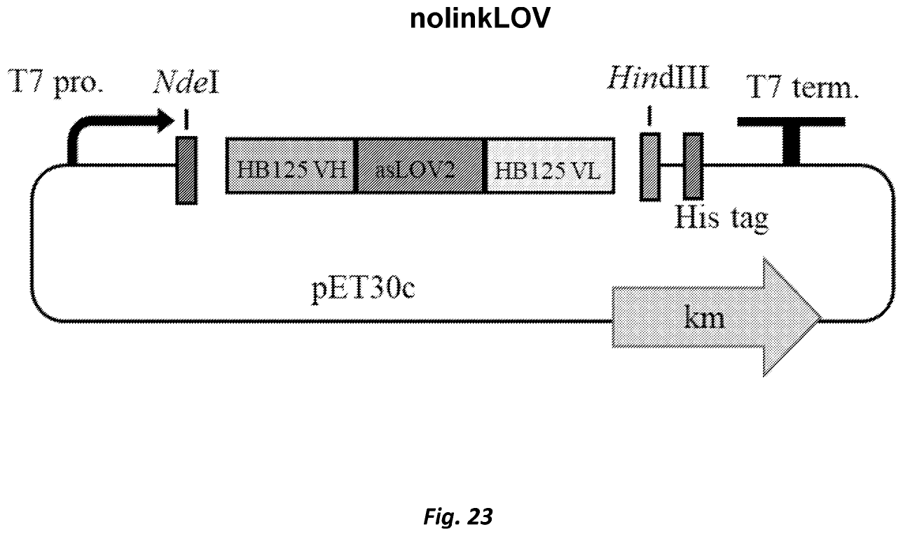

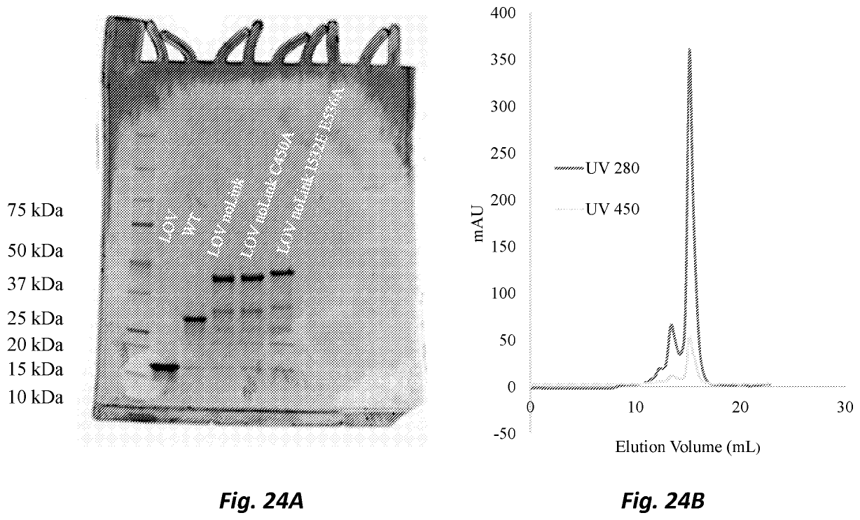

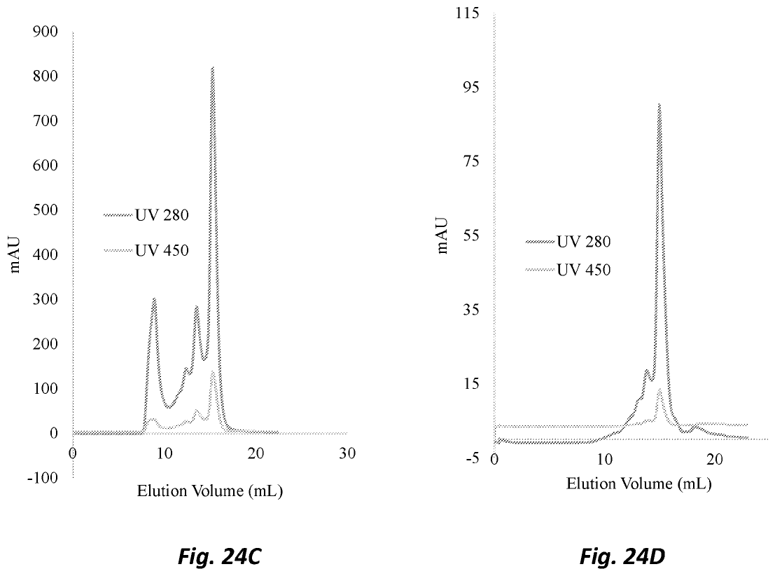

A fifth antibody fusion protein was designed with an asLOV2 domain between an anti-insulin V_{H} region at the N-terminus and an anti-insulin V_{L} region at the C-terminus without glycine-rich linkers between the domains (nolinkLOV). FIG. 22 shows the structure of the fifth engineered antibody fusion protein (nolinkLOV) as predicted by AlphaFold 2. The alpha helix involved in the conformational change of the asLOV2 domain is shown in dark gray. FIG. 23 shows a pET30c vector with NdeI and HindIII restriction sites and a His tag was used to produce the fifth recombinant antibody fusion protein (nolinkLOV) in E. coli Shuffle T7 Express™ cells (SEQ ID NO: 20). Variants of the nolinkLOV fusion proteins were prepared with mutations to fix the proteins in a “dark state” (C185A; SEQ ID NO: 29) or a “light state” (1267E, A271E; SEQ ID NO: 30). FIG. 24A shows SDS-PAGE of the eluted soluble nolinkLOV antibody fusion proteins expressed in E. coli Shuffle T7 Express™ cells. FIGS. 24B-24D show the chromatogram tracking at 280 nm (UV280) and 450 nm (UV450) absorptions for the nolinkLOV (FIG. 24B), the nolinkLOV C185A “dark” variant (FIG. 24C), and nolinkLOV I267E, A271E “light” variant (FIG. 24D) antibody fusion proteins.

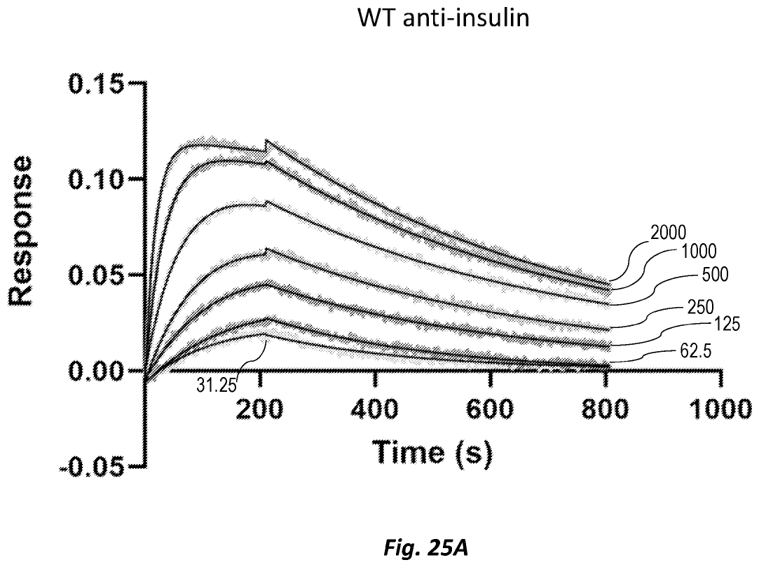

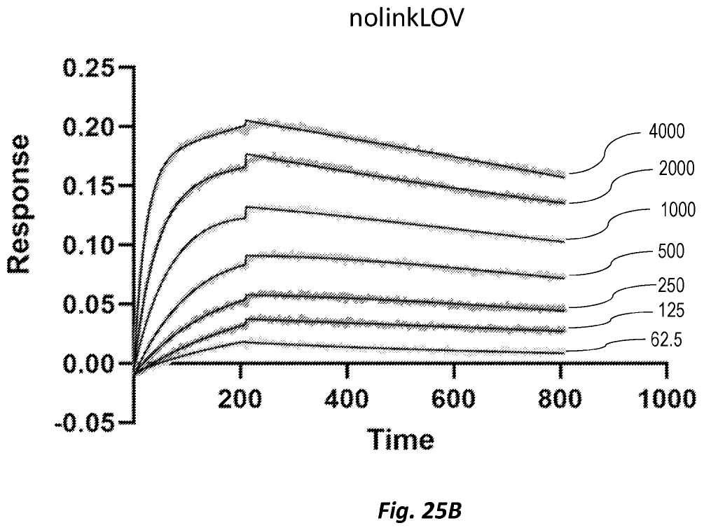

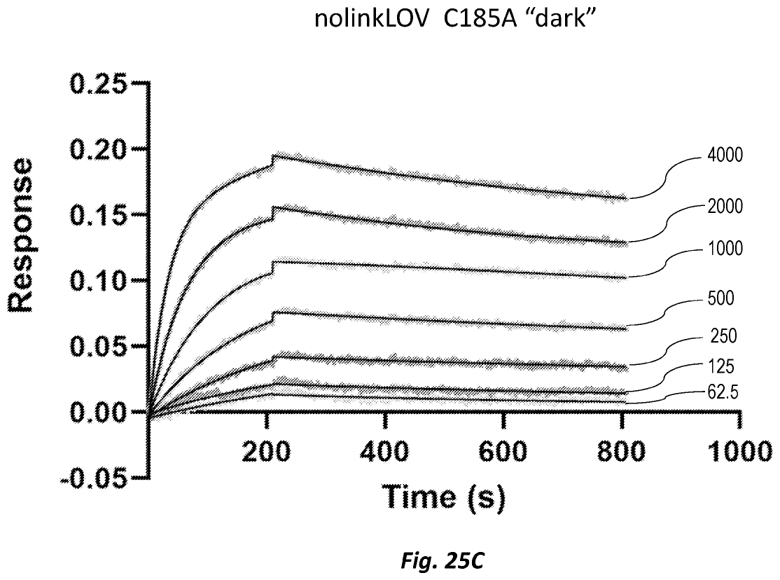

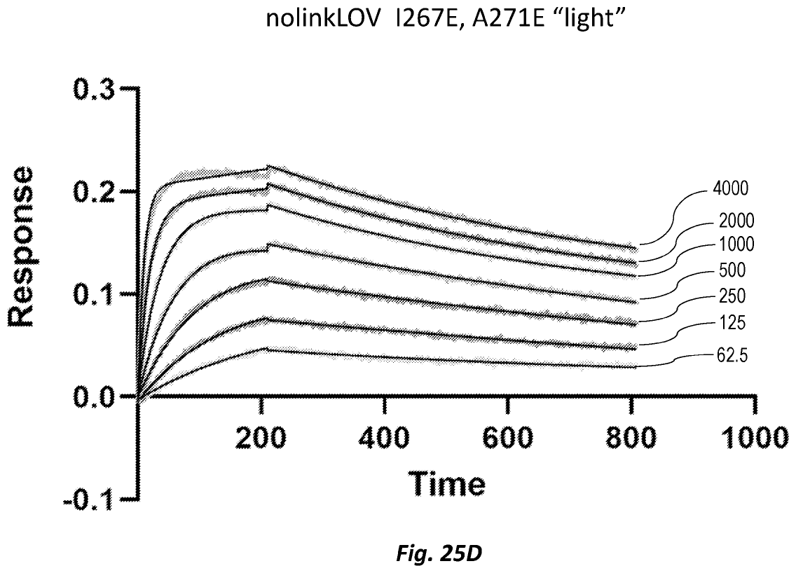

A Biolayer Interferometry (BLI) assay was performed to determine the binding affinity of the nolinkLOV antibody fusion proteins to insulin, as shown in FIG. 21A. FIGS. 25A-25D shows the results of the fifth antibody fusion binding to insulin via BLI (FIG. 21A). FIGS. 25A-25D show BLI sensor grams for wild type anti-insulin (FIG. 25A), the nolinkLOV fusion protein (FIG. 25B), the nolinkLOV C185A fusion protein fixed in a “dark state” (FIG. 25C) the nolinkLOV I267E, A271E fusion protein fixed in a “light state” (FIG. 25D). The corresponding calculated affinities shown in Table 2, indicating that the binding affinity of the dark mimetic fusion is almost four times that of the light mimetic fusion, which is comparable to the WT anti-insulin.

TABLE 2
Binding affinities of antibody fusion proteins with an asLOV2
domain between an anti-insulin V_{H} region at the N-terminus and
an anti-insulin V_{L} region at the C-terminus without
glycine-rich linkers (nolinkLOV)

| Sample | K_{D} (nM) | Kₒₙ (M⁻¹ * min-1) | K_{off} (min⁻¹) |
|---|---|---|---|
| WT anti-insulin (HB125) | 55.2 | 26790 | 0.001479 |
| nolinkLOV | 78.48 | 11236 | 0.0008819 |
| nolinkLOV C185A “dark” | 198 | 7333 | 0.001452 |
| nolinkLOV I267E, A271E “light” | 68.69 | 25802 | 0.001772 |

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. An engineered antibody fusion protein, the protein comprising a variable region of an immunoglobulin heavy chain (V_{H}), a variable region of an immunoglobulin light chain (V_{L}), and a redox-sensitive domain,
wherein the redox-sensitive domain is capable of undergoing a conformational change upon exposure to an external signal, and
wherein the conformational change modulates an affinity of the antibody fusion protein for a target molecule upon exposure to the external signal.

2. The antibody fusion protein of claim 1, wherein the amino acid sequence of the redox-sensitive domain is located at the C-terminal end of the antibody fusion protein.

3. The antibody fusion protein of claim 1, wherein the amino acid sequence of the redox-sensitive domain is located at the N-terminal end of the antibody fusion protein.

4. The antibody fusion protein of any one of claims 1-3, wherein the V_{H} region and the V_{L} region are adjacent and comprise a single chain variable region (scFv),
optionally wherein a linker sequence is between the amino acid sequence of the V_{H} region and the amino acid sequence of the V_{L} region, wherein the linker sequence comprises one or more glycine residues.

5. The antibody fusion protein of claim 1, wherein the amino acid sequence of the redox-sensitive domain is located between the amino acid sequence of the V_{H} region and the amino acid sequence of the V_{L} region.

6. The antibody fusion protein of claim 5, wherein the amino acid sequence of the V_{H} region is located at the C-terminal end of the antibody fusion protein and the amino acid sequence of the V_{L} region is located at the N-terminal end of the antibody fusion protein.

7. The antibody fusion protein of claim 5, wherein the amino acid sequence of the V_{H} region is located at the N-terminal end of the antibody fusion protein and the amino acid sequence of the V_{L} region is located at the C-terminal end of the antibody fusion protein.

8. The antibody fusion protein of any one of claims 1-7, wherein the target molecule is a metabolite, a hormone, and/or a therapeutic compound.

9. The antibody fusion protein of claim 8, wherein the target molecule is selected from the group consisting of insulin, glucagon, and glucagon-like peptide 1.

10. The antibody fusion protein of claim 9, wherein the target molecule is insulin.

11. The antibody fusion protein of claim 10, wherein the V_{H} region and the V_{L} region are anti-insulin.

12. The antibody fusion protein of claim 11, wherein the scFv comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 1.

13. The antibody fusion protein of claim 12, wherein the scFv comprises an amino acid sequence set forth in SEQ ID NO: 1.

14. The antibody fusion protein of any one of claims 1-13, wherein the redox-sensitive domain comprises a heme cofactor or a flavin cofactor.

15. The antibody fusion protein of any one of claims 1-14, wherein the redox-sensitive domain is selected from the group consisting of a bacterium-derived cytochrome c-like molecule (CYTc) domain, a light-oxygen-voltage-sensing (LOV) domain, and a cGMP-specific phosphodiesterases, adenylyl cyclases, FhlA (GAF) domain.

16. The antibody fusion protein of claim 15, wherein the LOV domain is a second LOV domain of a phototropin (LOV2).

17. The antibody fusion protein of claim 16, wherein the LOV2 domain is derived from Avena Sativa (asLOV2).

18. The antibody fusion protein of any one of claims 1-17, wherein the redox-sensitive domain comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 2 or SEQ ID NO: 36.

19. The antibody fusion protein of claim 18, wherein the redox-sensitive domain comprises an amino acid sequence set forth in SEQ ID NO: 2 or SEQ ID NO: 36.

20. The antibody fusion protein of any one of claims 1-19, wherein the amino acid sequence of the V_{H} region comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 6.

21. The antibody fusion protein of claim 20, wherein the amino acid sequence of the V_{H} region comprises an amino acid sequence set forth in SEQ ID NO: 6.

22. The antibody fusion protein of any one of claims 1-21, wherein the amino acid sequence of the V_{L} region comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to SEQ ID NO: 7.

23. The antibody fusion protein of claim 22, wherein the amino acid sequence of the V_{L} region comprises an amino acid sequence set forth in SEQ ID NO: 7.

24. The antibody fusion protein of any of claims 1-23, wherein the antibody fusion protein comprises an amino acid sequence having at least 90%, at least 91%, at least 92%, at least 93%, at least 94%, at least 95%, at least 96%, at least 97%, at least 98%, at least 99%, at least 99.5%, or at least 99.9% sequence identity to any one of SEQ ID NOs: 3-5, and 16-20.

25. The antibody fusion protein of claim 24, wherein the antibody fusion protein comprises an amino acid sequence set forth in any one of SEQ ID NO: 3-5, and 16-20.

26. The antibody fusion protein of any one of claims 1-25, wherein a sensitivity towards the target molecule is modulated by exposure to the external signal.

27. The antibody fusion protein of claim 26, wherein the sensitivity of the antibody fusion protein towards the target molecule is at least 2-fold, at least 5-fold, at least 10-fold, at least 100-fold, or at least 1000-fold different upon exposure to the external signal.

28. The antibody fusion protein of any one of claims 1-27, wherein a limit of detection (LOD) of the target molecule is modulated by exposure to the external signal.

29. The antibody fusion protein of any one of claims 1-28, wherein a specific binding ability of the antibody fusion protein towards the target molecule is modulated by exposure to the external signal.

30. The antibody fusion protein of claim 29, wherein the specific binding ability of the antibody fusion protein towards the target molecule is at least 2-fold, at least 5-fold, at least 10-fold, at least 100-fold, or at least 1000-fold different upon exposure to the external signal.

31. The antibody fusion protein of any one of claims 1-30, wherein the dissociation constant (K_{d}) of the antibody fusion protein towards the target molecule is modulated by exposure to the external signal.

32. The antibody fusion protein of claim 31, wherein the K_{d} of the antibody fusion protein is at least 10, at least 10², at least 10³, at least 10⁴, at least 10⁵, at least 10⁶, at least 10⁷, at least 10⁸, at least 10⁹, or at least 10¹⁰ times different upon exposure to the external signal.

33. The antibody fusion protein of any one of claims 1-32, wherein the external signal is redox potential or light.

34. The antibody fusion protein of claim 33, wherein the external signal is blue light.

35. The antibody fusion protein of claim 34, wherein the blue light has a wavelength of 400-500 nm.

36. An electrode comprising the antibody fusion protein of any of claims 1-35 immobilized on an electroconductive material.

37. A device for detection of the target molecule, comprising the engineered antibody fusion protein of any one of claims 1-35.

38. The device of claim 37, wherein the detection is continuous.

39. The device of claim 37 or 38, wherein the detection is performed in vivo.

40. The device of any one of claims 37-39, wherein the target molecule is insulin.

41. A polynucleotide encoding the antibody fusion protein of any one of claims 1-35.

42. The polynucleotide of claim 40, further comprising a nucleotide sequence having at least 90%, at least 91%, at least 92%, at least 93%, at least 94%, at least 95%, at least 96%, at least 97%, at least 98%, at least 99%, at least 99.5%, or at least 99.9% sequence identity to any one of SEQ ID NOs: 8-10, and 31-35.

43. The polynucleotide of claim 42, comprising a nucleotide sequence set forth in any one of SEQ ID NOs: 8-10, and 31-35.

44. A recombinant plasmid comprising a promoter operably linked to the polynucleotide sequence of any one of claims 41-43, and prepared by inserting the nucleotide sequence into an expression vector.

45. The recombinant plasmid of claim 44, wherein the expression vector is a pET30c vector.

46. A method of producing a recombinant antibody fusion protein, the method comprising:
i) introducing the recombinant plasmid of claim 44 or 45 into a host cell
ii) expressing the antibody fusion protein.

47. The method of claim 46, wherein the host cell is E. coli.


SEQUENCE LISTING (36 sequences)

SEQ ID NO: 1 - protein, 263 aa, synthetic construct
MAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGGGGSGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAA

SEQ ID NO: 2 - protein, 146 aa, Avena sativa
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLIKKTAEEIDEAAKEL

SEQ ID NO: 3 - protein, 414 aa, synthetic construct
MAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGGGGSGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAGGGGSGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLIKKTAEEIDEAAKEL

SEQ ID NO: 4 - protein, 411 aa, synthetic construct
MGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLIKKTAEEIDEAAGGGGSAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGGGGSGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAA

SEQ ID NO: 5 - protein, 401 aa, synthetic construct
MAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLIKKTAEEIDEAAGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAA

SEQ ID NO: 6 - protein, 134 aa, synthetic construct
AQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGS

SEQ ID NO: 7 - protein, 113 aa, synthetic construct
QIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAA

SEQ ID NO: 8 - DNA, 1023 bp, synthetic construct
  source 1..1023: mol_type = other DNA
atggcacagatccagttggtgcagtctggacctgaactgaagaagcctggagagacagtcaagatctcctgcaaggcttctggttataccttcacagactattcaatgcactgggtgaagcaggctccaggaaagggtttaaagtggatggactggataaacactgagactggtgtgccaacctatgcagatgacttcaagggacggtttgccttctctttggaaacctctgctaacactgcctatttgcagatcaacgacctcaaaaatgaggacacggctacatatttctgtactagagggtatggaaaggggtacttcgatgtctggggcgcagggaccacggtcaccgtctcctcagccaaaagcacacccccatccgtttatccattggcccctggaagcggcggaggcggatcaggaggaggaggatcaggcggaggaggatcacaaattgttctcacccagtctccaacaatcatgtctgcatctctaggggaacgggtcaccatgacctgcactgccagctcaagtgtaagttccagttacttgcactggtaccagcagaagccaggatcctcccccaaactctggatttatagtacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaacgggctgatgctgcaggcggaggcggatcaggcgaatttctggcgaccaccctggaacgcattgaaaaaaactttgtgattaccgatccgcgcctgccggataacccgattatttttgcgagcgatagctttctgcagctgaccgaatatagccgcgaagaaattctgggccgcaactgccgctttctgcagggcccggaaaccgatcgcgcgaccgtgcgcaaaattcgcgatgcgattgataaccag

SEQ ID NO: 9 - DNA, 1236 bp, synthetic construct
  source 1..1236: mol_type = other DNA
atgggcgaatttctggcgaccaccctggaacgcattgaaaaaaactttgtgattaccgatccgcgcctgccggataacccgattatttttgcgagcgatagctttctgcagctgaccgaatatagccgcgaagaaattctgggccgcaactgccgctttctgcagggcccggaaaccgatcgcgcgaccgtgcgcaaaattcgcgatgcgattgataaccagaccgaagtgaccgtgcagctgattaactataccaaaagcggcaaaaaattttggaacctgtttcatctgcagccgatgcgcgatcagaaaggcgatgtgcagtattttattggcgtgcagctggatggcaccgaacatgtgcgcgatgcggcggaacgcgaagcggtgatgctgattaaaaaaaccgcggaagaaattgatgaagcggcgaaagaactgggtagtggtgcacagatccagttggtgcagtctggacctgaactgaagaagcctggagagacagtcaagatctcctgcaaggcttctggttataccttcacagactattcaatgcactgggtgaagcaggctccaggaaagggtttaaagtggatggactggataaacactgagactggtgtgccaacctatgcagatgacttcaagggacggtttgccttctctttggaaacctctgctaacactgcctatttgcagatcaacgacctcaaaaatgaggacacggctacatatttctgtactagagggtatggaaaggggtacttcgatgtctggggcgcagggaccacggtcaccgtctcctcagccaaaagcacacccccatccgtttatccattggcccctggaagcggcggaggcggatcaggaggaggaggatcaggcggaggaggatcacaaattgttctcacccagtctccaacaatcatgtctgcatctctaggggaacgggtcaccatgacctgcactgccagctcaagtgtaagttccagttacttgcactggtaccagcagaagccaggatcctcccccaaactctggatttatagtacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaacgggctgatgctgca

SEQ ID NO: 10 - DNA, 1203 bp, synthetic construct
  source 1..1203: mol_type = other DNA
atggcacagatccagttggtgcagtctggacctgaactgaagaagcctggagagacagtcaagatctcctgcaaggcttctggttataccttcacagactattcaatgcactgggtgaagcaggctccaggaaagggtttaaagtggatggactggataaacactgagactggtgtgccaacctatgcagatgacttcaagggacggtttgccttctctttggaaacctctgctaacactgcctatttgcagatcaacgacctcaaaaatgaggacacggctacatatttctgtactagagggtatggaaaggggtacttcgatgtctggggcgcagggaccacggtcaccgtctcctcagccaaaagcacacccccatccgtttatccattggcccctggaagcggcggaggcggatcaggcgaatttctggcgaccaccctggaacgcattgaaaaaaactttgtgattaccgatccgcgcctgccggataacccgattatttttgcgagcgatagctttctgcagctgaccgaatatagccgcgaagaaattctgggccgcaactgccgctttctgcagggcccggaaaccgatcgcgcgaccgtgcgcaaaattcgcgatgcgattgataaccagaccgaagtgaccgtgcagctgattaactataccaaaagcggcaaaaaattttggaacctgtttcatctgcagccgatgcgcgatcagaaaggcgatgtgcagtattttattggcgtgcagctggatggcaccgaacatgtgcgcgatgcggcggaacgcgaagcggtgatgctgattaaaaaaaccgcggaagaaattgatgaagcggcgggcggaggaggatcacaaattgttctcacccagtctccaacaatcatgtctgcatctctaggggaacgggtcaccatgacctgcactgccagctcaagtgtaagttccagttacttgcactggtaccagcagaagccaggatcctcccccaaactctggatttatagtacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaacgggctgatgctgca

SEQ ID NO: 11 - DNA, 5423 bp, synthetic construct
  source 1..5423: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccacagatatcccatggccttgtcgtcgtcgtcggtacccagatctgggctgtccatgtgctggcgttcgaatttagcagcagcggtttctttcataccagaaccgcgtggcaccagaccagaagaatgatgatgatgatggtgcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 12 - DNA, 789 bp, synthetic construct
  source 1..789: mol_type = other DNA
atggcacagatccagttggtgcagtctggacctgaactgaagaagcctggagagacagtcaagatctcctgcaaggcttctggttataccttcacagactattcaatgcactgggtgaagcaggctccaggaaagggtttaaagtggatggactggataaacactgagactggtgtgccaacctatgcagatgacttcaagggacggtttgccttctctttggaaacctctgctaacactgcctatttgcagatcaacgacctcaaaaatgaggacacggctacatatttctgtactagagggtatggaaaggggtacttcgatgtctggggcgcagggaccacggtcaccgtctcctcagccaaaagcacacccccatccgtttatccattggcccctggaagcggcggaggcggatcaggaggaggaggatcaggcggaggaggatcacaaattgttctcacccagtctccaacaatcatgtctgcatctctaggggaacgggtcaccatgacctgcactgccagctcaagtgtaagttccagttacttgcactggtaccagcagaagccaggatcctcccccaaactctggatttatagtacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaacgggctgatgctgca

SEQ ID NO: 13 - DNA, 405 bp, synthetic construct
  source 1..405: mol_type = other DNA
atggcacagatccagttggtgcagtctggacctgaactgaagaagcctggagagacagtcaagatctcctgcaaggcttctggttataccttcacagactattcaatgcactgggtgaagcaggctccaggaaagggtttaaagtggatggactggataaacactgagactggtgtgccaacctatgcagatgacttcaagggacggtttgccttctctttggaaacctctgctaacactgcctatttgcagatcaacgacctcaaaaatgaggacacggctacatatttctgtactagagggtatggaaaggggtacttcgatgtctggggcgcagggaccacggtcaccgtctcctcagccaaaagcacacccccatccgtttatccattggcccctggaagc

SEQ ID NO: 14 - DNA, 339 bp, synthetic construct
  source 1..339: mol_type = other DNA
caaattgttctcacccagtctccaacaatcatgtctgcatctctaggggaacgggtcaccatgacctgcactgccagctcaagtgtaagttccagttacttgcactggtaccagcagaagccaggatcctcccccaaactctggatttatagtacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaacgggctgatgctgca

SEQ ID NO: 15 - DNA, 438 bp, Avena sativa
  source 1..438: mol_type = other DNA
ggcgaatttctggcgaccaccctggaacgcattgaaaaaaactttgtgattaccgatccgcgcctgccggataacccgattatttttgcgagcgatagctttctgcagctgaccgaatatagccgcgaagaaattctgggccgcaactgccgctttctgcagggcccggaaaccgatcgcgcgaccgtgcgcaaaattcgcgatgcgattgataaccagaccgaagtgaccgtgcagctgattaactataccaaaagcggcaaaaaattttggaacctgtttcatctgcagccgatgcgcgatcagaaaggcgatgtgcagtattttattggcgtgcagctggatggcaccgaacatgtgcgcgatgcggcggaacgcgaagcggtgatgctgattaaaaaaaccgcggaagaaattgatgaagcggcgaaagaactg

SEQ ID NO: 16 - protein, 409 aa, synthetic construct
MAQIQLVQSGPELKKPGETVKISCKASGYTFTDYSMHWVKQAPGKGLKWMDWINTETGVPTYADDFKGFAFSLETSANTAYLQINDLKNEDTATYFCTRGYGKGYFDVWGAGTTVTVSSAKSTPPSVYPLAPGSGGGGSGGGGSGGGGSQIVLTQSPTIMSASLGERVTTCTASSSVSSSYLHWYQQKPGSSPKLWIYSTSNLASGVPARFSGSGSGTSYSLTISSMEAEDAATYYCHQYHRSPPTFGAGTKLELKRADAAGGGGGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGQIGVQLDGTEHVRDAAEREAVMLIKKTAEEIDEAAKELKL

SEQ ID NO: 17 - protein, 411 aa, synthetic construct
MGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLIKKTAEEIDEAAKELAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGGGGSGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 18 - protein, 401 aa, synthetic construct
MGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLIKKTAAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGGGGSGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 19 - protein, 406 aa, synthetic construct
MAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLIKKTAEEIDEAAKELGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 20 - protein, 396 aa, synthetic construct
MAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLIKKTAEEIDEAAKELQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 21 - protein, 414 aa, synthetic construct
MAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGGGGSGGGGSQIVLTQSPTIMSASLGERVTMTCTASSSVSSSYLHWYQKPGSSPKLWIYSTSNLASGVPARFSGSGSGTSYSLTISSMEAEDAATYYCHQYHRSPPTFGAGTKLELKRADAAGGGGSGEFLATLERIEKNFVITDPRLPDNPIIFASDSFLQLTEYSREEILGRNARFLQGPETDRATVRKIRDAIDNQTEVTVQLINYTKSGKKFWNLFHLQPMRDQKGDVQYFIGVQLDGTEHVRDAAEREAVMLIKKTAEEIDEAAKELKL

SEQ ID NO: 22 - protein, 416 aa, synthetic construct
MAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGGGGSGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAGGGGSGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLEKKTEEEIDEAAKELKL

SEQ ID NO: 23 - protein, 411 aa, synthetic construct
MGEFLATTLERIEKNFVITDPRLPDNPIIFASDSFLQLTEYSREEILGRNARFLQGPETDRATVRKIRDAIDNQTEVTVQLINYTKSGKKFWNLFHLQPMRDQKGDVQYFIGVQLDGTEHVRDAAEREAVMLIKKTAEEIDEAAKELAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGGGGSGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 24 - protein, 411 aa, synthetic construct
MGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLEKKTEEEIDEAAKELAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGGGGSGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 25 - protein, 399 aa, synthetic construct
MGEFLATTLERIEKNFVITDPRLPDNPIIFASDSFLQLTEYSREEILGRNARFLQGPETDRATVRKIRDAIDNQTETVQLINYTKSGKKFWNLFHLQPMRDQKGDVQYFIGVQLDGTEHVRDAAEREAVMLIKKTAAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGGGGSGGGGSQIVLTSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 26 - protein, 401 aa, synthetic construct
MGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLEKKTEAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGGGGSGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 27 - protein, 406 aa, synthetic construct
MAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGEFLATTLERIEKNFVITDPRLPDNPIIFASDSFLQLTEYSREEILGRNARFLQGPETDRATVRKIRDAIDNQTEVTVQLINYTKSGKKFWNLFHLQPMRDQKGDVQYFIGVQLDGTEHVRDAAEREAVMLIKKTAEEIDEAAKELGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 28 - protein, 406 aa, synthetic construct
MAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGGGGSGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLEKKTEEEIDEAAKELGGGGSQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 29 - protein, 396 aa, synthetic construct
MAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGEFLATTLERIEKNFVITDPRLPDNPIIFASDSFLQLTEYSREEILGRNARFLQGPETDRATVRKIRDAIDNQTEVTVQLINYTKSGKKFWNLFHLQPMRDQKGDVQYFIGVQLDGTEHVRDAAEREAVMLIKKTAEEIDEAAKELQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 30 - protein, 396 aa, synthetic construct
MAQIQLVQSGPELKKPGETVKISCKASGYTFTDYSMHWVKQAPGKGLKWMDWINTETGVPTYADDFKGRFAFSLETSANTAYLQINDLKNEDTATYFCTRGYGKGYFDVWGAGTTVTVSSAKSTPPSVYPLAPGSGEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLEKKTEEEIDEAAKELQIVLTQSPTIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELKRADAAKL

SEQ ID NO: 31 - DNA, 1251 bp, synthetic construct
  source 1..1251: mol_type = other DNA
catatggcacagatccagttggtgcagtctggacctgaactgaagaagcctggagagacagtcaagatctcctgcaaggcttctggttataccttcacagactattcaatgcactgggtgaagcaggctccaggaaagggtttaaagtggatggactggataaacactgagactggtgtgccaacctatgcagatgacttcaagggacggtttgccttctctttggaaacctctgctaacactgcctatttgcagatcaacgacctcaaaaatgaggacacggctacatatttctgtactagagggtatggaaaggggtacttcgatgtctggggcgcagggaccacggtcaccgtctcctcagccaaaagcacacccccatccgtttatccattggcccctggaagcggcggaggcggatcaggaggaggaggatcaggcggaggaggatcacaaattgttctcacccagtctccaacaatcatgtctgcatctctaggggaacgggtcaccatgacctgcactgccagctcaagtgtaagttccagttacttgcactggtaccagcagaagccaggatcctcccccaaactctggatttatagtacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaacgggctgatgctgcaggcggaggcggatcaggcgaatttctggcgaccaccctggaacgcattgaaaaaaactttgtgattaccgatccgcgcctgccggataacccgattatttttgcgagcgatagctttctgcagctgaccgaatatagccgcgaagaaattctgggccgcaactgccgctttctgcagggcccggaaaccgatcgcgcgaccgtgcgcaaaattcgcgatgcgattgataaccagaccgaagtgaccgtgcagctgattaactataccaaaagcggcaaaaaattttggaacctgtttcatctgcagccgatgcgcgatcagaaaggcgatgtgcagtattttattggcgtgcagctggatggcaccgaacatgtgcgcgatgcggcggaacgcgaagcggtgatgctgattaaaaaaaccgcggaagaaattgatgaagcggcgaaagaactgaagctt

SEQ ID NO: 32 - DNA, 1236 bp, synthetic construct
  source 1..1236: mol_type = other DNA
catatgggcgaatttctggcgaccaccctggaacgcattgaaaaaaactttgtgattaccgatccgcgcctgccggataacccgattatttttgcgagcgatagctttctgcagctgaccgaatatagccgcgaagaaattctgggccgcaactgccgctttctgcagggcccggaaaccgatcgcgcgaccgtgcgcaaaattcgcgatgcgattgataaccagaccgaagtgaccgtgcagctgattaactataccaaaagcggcaaaaaattttggaacctgtttcatctgcagccgatgcgcgatcagaaaggcgatgtgcagtattttattggcgtgcagctggatggcaccgaacatgtgcgcgatgcggcggaacgcgaagcggtgatgctgattaaaaaaaccgcggaagaaattgatgaagcggcgaaagaactggcacagatccagttggtgcagtctggacctgaactgaagaagcctggagagacagtcaagatctcctgcaaggcttctggttataccttcacagactattcaatgcactgggtgaagcaggctccaggaaagggtttaaagtggatggactggataaacactgagactggtgtgccaacctatgcagatgacttcaagggacggtttgccttctctttggaaacctctgctaacactgcctatttgcagatcaacgacctcaaaaatgaggacacggctacatatttctgtactagagggtatggaaaggggtacttcgatgtctggggcgcagggaccacggtcaccgtctcctcagccaaaagcacacccccatccgtttatccattggcccctggaagcggcggaggcggatcaggaggaggaggatcaggcggaggaggatcacaaattgttctcacccagtctccaacaatcatgtctgcatctctaggggaacgggtcaccatgacctgcactgccagctcaagtgtaagttccagttacttgcactggtaccagcagaagccaggatcctcccccaaactctggatttatagtacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaacgggctgatgctgcaaagctt

SEQ ID NO: 33 - DNA, 1206 bp, synthetic construct
  source 1..1206: mol_type = other DNA
catatgggcgaatttctggcgaccaccctggaacgcattgaaaaaaactttgtgattaccgatccgcgcctgccggataacccgattatttttgcgagcgatagctttctgcagctgaccgaatatagccgcgaagaaattctgggccgcaactgccgctttctgcagggcccggaaaccgatcgcgcgaccgtgcgcaaaattcgcgatgcgattgataaccagaccgaagtgaccgtgcagctgattaactataccaaaagcggcaaaaaattttggaacctgtttcatctgcagccgatgcgcgatcagaaaggcgatgtgcagtattttattggcgtgcagctggatggcaccgaacatgtgcgcgatgcggcggaacgcgaagcggtgatgctgattaaaaaaaccgcggcacagatccagttggtgcagtctggacctgaactgaagaagcctggagagacagtcaagatctcctgcaaggcttctggttataccttcacagactattcaatgcactgggtgaagcaggctccaggaaagggtttaaagtggatggactggataaacactgagactggtgtgccaacctatgcagatgacttcaagggacggtttgccttctctttggaaacctctgctaacactgcctatttgcagatcaacgacctcaaaaatgaggacacggctacatatttctgtactagagggtatggaaaggggtacttcgatgtctggggcgcagggaccacggtcaccgtctcctcagccaaaagcacacccccatccgtttatccattggcccctggaagcggcggaggcggatcaggaggaggaggatcaggcggaggaggatcacaaattgttctcacccagtctccaacaatcatgtctgcatctctaggggaacgggtcaccatgacctgcactgccagctcaagtgtaagttccagttacttgcactggtaccagcagaagccaggatcctcccccaaactctggatttatagtacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaacgggctgatgctgcaaagctt

SEQ ID NO: 34 - DNA, 1221 bp, synthetic construct
  source 1..1221: mol_type = other DNA
catatggcacagatccagttggtgcagtctggacctgaactgaagaagcctggagagacagtcaagatctcctgcaaggcttctggttataccttcacagactattcaatgcactgggtgaagcaggctccaggaaagggtttaaagtggatggactggataaacactgagactggtgtgccaacctatgcagatgacttcaagggacggtttgccttctctttggaaacctctgctaacactgcctatttgcagatcaacgacctcaaaaatgaggacacggctacatatttctgtactagagggtatggaaaggggtacttcgatgtctggggcgcagggaccacggtcaccgtctcctcagccaaaagcacacccccatccgtttatccattggcccctggaagcggcggaggcggatcaggcgaatttctggcgaccaccctggaacgcattgaaaaaaactttgtgattaccgatccgcgcctgccggataacccgattatttttgcgagcgatagctttctgcagctgaccgaatatagccgcgaagaaattctgggccgcaactgccgctttctgcagggcccggaaaccgatcgcgcgaccgtgcgcaaaattcgcgatgcgattgataaccagaccgaagtgaccgtgcagctgattaactataccaaaagcggcaaaaaattttggaacctgtttcatctgcagccgatgcgcgatcagaaaggcgatgtgcagtattttattggcgtgcagctggatggcaccgaacatgtgcgcgatgcggcggaacgcgaagcggtgatgctgattaaaaaaaccgcggaagaaattgatgaagcggcgaaagaactgggcggaggcggatcacaaattgttctcacccagtctccaacaatcatgtctgcatctctaggggaacgggtcaccatgacctgcactgccagctcaagtgtaagttccagttacttgcactggtaccagcagaagccaggatcctcccccaaactctggatttatagtacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaacgggctgatgctgcaaagctt

SEQ ID NO: 35 - DNA, 1191 bp, synthetic construct
  source 1..1191: mol_type = other DNA
catatggcacagatccagttggtgcagtctggacctgaactgaagaagcctggagagacagtcaagatctcctgcaaggcttctggttataccttcacagactattcaatgcactgggtgaagcaggctccaggaaagggtttaaagtggatggactggataaacactgagactggtgtgccaacctatgcagatgacttcaagggacggtttgccttctctttggaaacctctgctaacactgcctatttgcagatcaacgacctcaaaaatgaggacacggctacatatttctgtactagagggtatggaaaggggtacttcgatgtctggggcgcagggaccacggtcaccgtctcctcagccaaaagcacacccccatccgtttatccattggcccctggaagcggcgaatttctggcgaccaccctggaacgcattgaaaaaaactttgtgattaccgatccgcgcctgccggataacccgattatttttgcgagcgatagctttctgcagctgaccgaatatagccgcgaagaaattctgggccgcaactgccgctttctgcagggcccggaaaccgatcgcgcgaccgtgcgcaaaattcgcgatgcgattgataaccagaccgaagtgaccgtgcagctgattaactataccaaaagcggcaaaaaattttggaacctgtttcatctgcagccgatgcgcgatcagaaaggcgatgtgcagtattttattggcgtgcagctggatggcaccgaacatgtgcgcgatgcggcggaacgcgaagcggtgatgctgattaaaaaaaccgcggaagaaattgatgaagcggcgaaagaactgcaaattgttctcacccagtctccaacaatcatgtctgcatctctaggggaacgggtcaccatgacctgcactgccagctcaagtgtaagttccagttacttgcactggtaccagcagaagccaggatcctcccccaaactctggatttatagtacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaacgggctgatgctgcaaagctt

SEQ ID NO: 36 - protein, 136 aa, synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREAVMLIKKTA